(19) United States
(12) Patent Application Publication
(10) Pub. No.: US 2012/0108878 A1
(43) Pub. Date: May 3, 2012

(54) Novel Chemistries, Solutions, and Dispersal Systems for Decontamination of Chemical and Biological Systems

(75) Inventor: Michael J. Conrad, Escondido, CA (US)

(73) Assignee: Aries Associates, Inc., Escondido, CA (US)

(21) Appl. No.: 12/631,599

(22) Filed: Dec. 4, 2009

Related U.S. Application Data
(63) Continuation of application No. 12/567,604, filed on Sep. 25, 2009.
(60) Provisional application No. 61/112,689, filed on Nov. 7, 2008.
(60) Provisional application No. 61/116,627, filed on Nov. 20, 2008.

(51) Int. Cl. A62D 3/38; A61L 2/18; A01P 3/00; A01N 37/18; A01P 1/00

(52) U.S. Cl. 588/299; 514/616; 510/110; 422/292; 588/320

(57) ABSTRACT

The present invention relates generally to chemical and biological decontamination solutions and methods of using them. The invention is useful for decontaminating a wide range of compounds and organisms. In particular, the systems, methods, solutions, and formulations of the invention can be used to remove and/or neutralize organophosphates and other toxic chemicals, bacteria, bacterial spores, fungi, molds and viruses.

This application is a continuation of U.S. patent application Ser. No. 12/567,604, filed Sep. 25, 2009, which application claims benefit of priority to U.S. Provisional Patent Application No. 61/112,689, filed on Nov. 7, 2008, and U.S. Provisional Patent Application No. 61/116,627, filed on Nov. 20, 2008. Each of these applications are incorporated by reference in their entirety, including any disclosure and references therein.

### FIELD OF THE INVENTION

The present invention relates generally to chemical and biological decontamination solutions and methods of using them. The invention is useful for decontaminating a wide range of compounds and organisms by reducing them to harmless, environmentally safe by-products. In particular, the methods, solutions, and formulations of the invention can be used to neutralize organophosphates, mustard agents and other toxic chemicals, bacteria, bacterial spores, fungi, molds and viruses.

### BACKGROUND OF THE INVENTION

Terrorist threats based on the use of chemical and biological toxants are increasing both in the United States and abroad. The use, and threat of use, of chemical and biological agents in the context of weapons of mass destruction are of paramount concern both to national defense as well as to state and local law enforcement. The threats from chemical toxants and biopathogens are not restricted to terrorism, however. Chemical pollution of water resources is one of the major threats to sustainable water resources development and management. Chemical pollution can be caused by: poorly treated or untreated municipal and industrial wastewater; pesticide and fertilizer run-off from agriculture; spills and other ship-related releases; mining; and other sources. Communicable pathogens like Influenza A (H1N1), Bacillus anthracia (anthrax), Yersinia pestis (plague) and Mycobacterium tuberculosis (TB) have the potential to spread quickly across the planet, and to create global pandemics as the result of international travel by air travel, ships and even routine cross border travel on public transit.

All of these threats can be referred to by the term “toxants,” which includes both toxic chemical compounds and biological entities, including, but not limited to, pesticides, blister agents, nerve agents, and biopathogens (e.g., bacteria, bacterial spores, viruses, and toxins). If left without decontamination, toxants can cause death, incapacitation, or permanent harm to humans, animals, or other organisms. Moreover, failure to disinfect to safe levels of communicable pathogens as influenza viruses, bacterial spores and vegetative bacteria can lead to the pandemic spread of infectious diseases.

Certain chemical toxants, including chemical warfare (“CW”) agents known to pose a threat by terrorists, share chemical characteristics that, in the case of the present patent, present an opportunity to develop a general theory of decontamination on the basis of which novel countermeasures as disclosed herein have been created.

G-class nerve agents like sarin (GB), soman (GD), and tabun (GA), are examples of extremely toxic organophosphate ester derivatives which, when chemically altered, can lose their toxicity, but can also be converted into other undesirable toxic compounds. The G-agents can also be volatile and present vapor hazards.

V-class agents are also organophosphate esters, with VX (S-[2-(diisopropylamino)ethyl]-O-ethyl methylphosphonothioate) being the most widely known and deployed. Mustard agents, (which are “blister agents”), of which HD (bis-(2-chloroethyl) sulfide or 1,1′-thiobis(2-chloroethane)) is the most widely known, are not nerve agents or organophosphates, but are CW agents that can be rendered harmless by chemical oxidation under limited circumstances. The stabilities, water solubilities and vapor pressures of these agents can vary. V-agents tend to be persistent on surfaces.

Certain of the known biological warfare (“BW”) agents include Bacillus anthracis (anthrax) and other spore-forming bacteria, non-sporulating but pathogenic bacteria, including Yersinia pestis (plague), and various enveloped and non-enveloped viruses, can be deactivated chemically. Together, these types of toxants are commonly referred to as “CBW” agents.

A CBW attack, infectious disease outbreak, or accident can involve either local placement or wide dispersal of the agent or agents so as to affect a population of human individuals. Because of the flexibility with which CBW agents can be deployed, respondents might encounter the agents in a variety of physical states including liquids, aerosols, and vapors.

An effective, rapid, and safe (i.e., non-toxic and non-corrosive) decontamination technology is desired in the event of a domestic terrorist attack, a chemical accident, or a biological pandemic. Decontamination includes substantially complete neutralization and/or substantially complete destruction of the chemical warfare or biological warfare agents. Ideally, technology is desired that would be applicable to a variety of scenarios such as the decontamination of open, semi-enclosed, and enclosed facilities as well as sensitive equipment. Examples of types of facilities where the decontamination formulation may be utilized include a stadium (open), an underground subway station (semi-enclosed), and an airport terminal or office building (enclosed).

Many essential biochemicals are organophosphates, including DNA, RNA, phospholipids and many essential cofactors. In health, agriculture, and military applications, the word “organophosphates” refers to a subset of all organophosphates that act as insecticides or nerve agents by inhibiting the enzyme acetylcholinesterase, which converts acetylcholine to choline and acetate. Acetylcholine is a neurotransmitter found in both the peripheral nervous systems (PNS) and central nervous systems (CNS) of many organisms, including humans, which is distinguished by its actions on cholinergic receptors (“cholinergic” actions) at the neuromuscular junction connecting motor nerves to muscles. The parasympathetic nervous system is entirely cholinergic. Neuromuscular junctions, preganglionic neurons of the sympathetic nervous system, the basal forebrain, and brain stem complexes are also cholinergic. The paralytic arrow-poison curare is a naturally occurring nerve agent which acts by blocking transmission at these synapses.

Acetylcholinesterase is abundant in the synaptic cleft, and its role in rapidly clearing free acetylcholine from the synapse is essential for proper muscle function. Organophosphate toxants work by inhibiting acetylcholinesterase, leading to excess acetylcholine at the neuromuscular junction which can, in turn, cause paralysis of the muscles needed for breathing and stopping the beating of the heart. Many organophosphates have neurotoxic effects on developing organisms, even at low exposure levels. Their toxicity is not limited to an acute phase, however, and chronic effects have long been noted.

Some organophosphate compounds are also potent pesticides and are classified as weapons of mass destruction by the United Nations according to UN Resolution 687 (passed in April 1991). Their production and stockpiling was outlawed by the Chemical Weapons Convention of 1993, which officially took effect on Apr. 29, 1997.

The term “organophosphate” has, in recent years, also been used more generically to describe virtually any organic phosphorus (V)-containing compound, especially when dealing with neurotoxic compounds. Many compounds that are included within the organophosphate class actually contain carbon-phosphate bonds. For instance, the nerve agent sarin has the IUPAC name O-isopropyl methylphosphonofluoridate, and is derived from phosphorous acid (HP(O)(OH)₂), rather than phosphoric acid (P(O)(OH)₃). Also, many compounds that are derivatives of phosphinic acid are used as neurotoxic organophosphates. Organophosphate pesticides, as well as sarin and the VX nerve agent, irreversibly inactivate acetylcholinesterase, which is essential to nerve function in insects, humans, and many other animals. Organophosphate pesticides affect this enzyme in varied ways, and thus vary in their potential for poisoning. For example, parathion, one of the first organophosphates commercialized, is many times more potent than malathion, an insecticide used in combating the Mediterranean fruit fly (Med-fly) and West Nile Virus-transmitting mosquitoes.

Many, but not all, organophosphate pesticides degrade rapidly by hydrolysis on exposure to sunlight, air, and soil. However, small amounts of these compounds can still be detected in food and drinking water. The ability to degrade over time has made them an attractive alternative to the persistent organochloride pesticides, such as DDT, aldrin, and dieldrin. Although organophosphates degrade faster than the organochlorides, they have greater acute toxicity, posing risks to people who may be exposed to large amounts of these compounds. Common organophosphates which have been used as pesticides include parathion, malathion, methyl parathion, chlorpyrifos, diazinon, dichlorvos, phosmet, tetrachlorvinphos, and azinphos methyl.

VX is an extremely toxic organophosphate and is so dangerous, even in extremely small volumes, that its only application is in chemical warfare as a nerve agent. VX is also the most toxic of the deployed chemical weapons and is classified as a weapon of mass destruction by the United Nations in UN Resolution 687. Like other organophosphorus nerve agents, VX may be destroyed by reaction with strong nucleophiles such as pralidoxime. The reaction of VX with concentrated aqueous sodium hydroxide results in competing cleavage of P—O and P—S esters, with P—S cleavage dominating. This can be a problem when hydrogen peroxide is used as a decontaminant, since one by-product of P—O bond cleavage (named EA 2192) is nearly as toxic as VX itself and is far more persistent in the environment. In contrast, reaction with the anion of percarboxylic acids (perhydrolysis) leads to exclusive cleavage of the P—S bond:

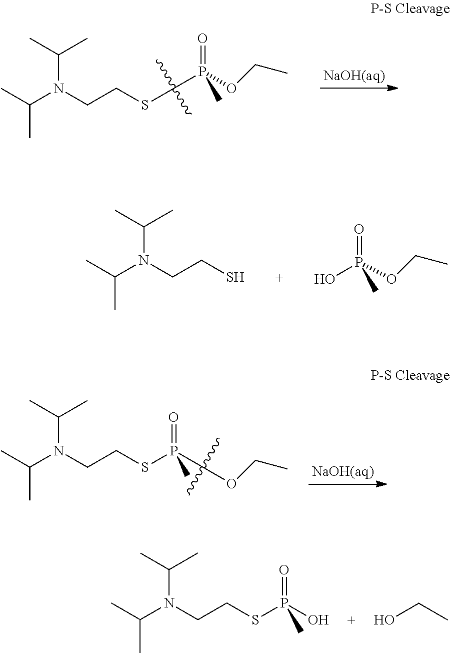

The H-class (known as “blister agents” or “sulfur mustards”) are not organophosphates. Rather, these compounds are a class of cytotoxic, vesicant CBW agents with the ability to form large blisters on exposed skin. Vesicants are highly active corrosive materials, even at extremely low concentrations. Compounds of this type comprise the structural element BCH₂CH₂X, where B is any leaving group and X is a Lewis base. Such compounds can form cyclic “onium” ions (sulfonium, ammoniums, etc.), which are good alkylating agents. Examples of blister agents include, but are not limited to, bis(2-chloroethyl)ether, the (2-haloethyl) amines (nitrogen mustards) and sulfur sesquimustard, which has two β-chloroethyl thioether groups (ClH₂C—CH₂—S—) connected by an ethylene (—CH₂CH₂—) group. These compounds have a similar ability to alkylate DNA, but their physical properties, e.g., melting point, can vary considerably. Some of these compounds have melting points well below the freezing point of water. The most well known of these compounds is commonly referred to as “mustard gas” (bis-(2-chloroethyl) sulfide or 1,1′-thiobis(2-chloroethane)), and, in its pure form, is a colorless, odorless, viscous liquid designated as HD, which is a β-chloro thioether with the formula C₄H₈Cl₂S. HD is a liquid at room temperature and has melting point of 14° C. (57° F.).

These vesicant agents can be quite deadly as they have a high solubility in lipids (e.g., fatty tissues). Symptoms of exposure to mustard gas include conjunctivitis, blindness, cough, edema of the eyelids, and erythema or necrosis of the skin. When inhaled, this can severely and irreparably damage the respiratory tract. In addition, mustard gas is also a carcinogen. Vesicants have other uses besides chemical warfare, however, the vesicating properties of these compounds are an undesirable/unwanted side effect. For example, some chemotherapy drugs are mild vesicants, as are a variety of industrially useful chemical intermediates.

The term “biopathogen” encompasses CBW agents that are infectious biological agents which can cause disease or illness to a host. These biopathogens include, but are not limited to, bacteria, bacterial spores, viruses, molds, fungi, and their toxins. Pathogenic bacteria can cause infectious diseases; the most common bacterial disease is tuberculosis, which is caused by the bacterium Mycobacterium tuberculosis. Mycobacterium is a genus of Actinobacteria, which includes many pathogens known to cause serious diseases in mammals, including tuberculosis and leprosy. Pathogenic bacteria also contribute to other globally important diseases, such as pneumonia, which can be caused by bacteria such as Streptococcus and Pseudomonas, and foodborne illnesses, which can be caused by bacteria such as Shigella, Campylobacter, and Salmonella. Pathogenic bacteria also cause infections such as tetanus, typhoid fever, diphtheria, syphilis and leprosy. Pathogenic viruses are mainly those of the families of: Adenoviridae, Picornaviridae, Herpesviridae, Hepadnaviridae, Flaviviridae, Retroviridae, Orthomyxoviridae, Paramyxoviridae, Papovaviridae, Rhabdoviridae, Togaviridae. Some notable pathogenic viruses cause smallpox, influenza, mumps, measles, chickenpox, and rubella.

The threat from biological toxants can be even more serious than the chemical warfare threat. This is in part because of the high toxicity of BW agents, their ease of acquisition and production, and their difficulty in detection but also, as in the case of pandemics, their ease of transmission and spread. There are hundreds of biological warfare toxants known, with new viruses appearing constantly. For decontamination purposes, biological toxants can be usefully distinguished as spore forming bacteria which can exist in a vegetative state (e.g., Bacillus anthracis which causes anthrax), bacteria which are vegetative but non-sporulating (e.g., Yersinia pestis the cause of plague, Vibrio cholerae the cause of cholera), non-lipid and small viruses (e.g., polio viruses), fungi (e.g., Trichophyton spp.), lipid and medium size viruses (e.g., retroviruses like HIV, Hepatitis B viruses), and bacterial toxins (e.g., botulism, ricin).

With the exception of prions, bacterial spores are recognized to be the most difficult microorganism to kill. Prions are infectious agents composed of protein which propagate by transmitting in a mis-folded protein state, and are not generally considered biological warfare agents. Bacterial spores are highly resistant structures formed by certain gram-positive bacteria usually in response to stresses in their environment. The most important spore-formers are members of the genera Bacillus (e.g., Bacillus anthracis) and Clostridium (e.g., Clostridium difficile). Spores are considerably more complex than vegetative cells. The outer surface of a spore consists of the spore coat that is typically made up of a dense layer of insoluble proteins usually containing a large number of disulfide bonds. The cortex consists of peptidoglycan, a polymer primarily made up of highly crosslinked N-acetylglucosamine and N-acetylmuramic acid. The spore core contains normal (vegetative) cell structures such as ribosomes and a nucleoid.

Antiseptics and disinfectants are used extensively in hospitals and other health care settings for a variety of topical and hard-surface applications to deal with biological toxants. In particular, they are an essential part of infection control practices and aid in the prevention of nosocomial infections. There are a variety of sterilants and disinfectants that can be used to address decontamination of one or more biological pathogens, as shown in Table 1.

TABLE 1
Mechanisms of antibacterial actions of disinfectants and sterilants

| Target | Disinfectant | Mechanism of Action |
|---|---|---|
| Cell wall, | Glutaraldehyde | Cross-linking of proteins |
| outer | EDTA, other | Gram-negative bacteria: removal of |
| membrane | permeabilizers | Mg²⁺, release of some LPS |
| Cyto- | QACs | Generalized membrane damage involving |
| plasmic | | phospholipid bilayers |
| membrane | Chlorhexidine | Low concentrations affect membrane |
| | | integrity, high concentrations |
| | | cause congealing of cytoplasm |
| | Diamines | Induction of leakage of amino acids |
| | PHMB, | Phase separation and domain formation of |
| | Alexidine | membrane lipids |
| | Phenols | Leakage; some cause uncoupling |
| Cross- | Formal- | Cross-linking of proteins, RNA, and DNA |
| linking of | dehyde | |
| macro- | Glutaraldehyde | Cross-linking of proteins in cell envelope |
| molecules | | and elsewhere in the cell |
| DNA | Acridines | Intercalation of an acridine molecule |
| intercalation | | between two layers of base pairs in DNA |
| Interaction | Silver | Membrane-bound enzymes (interaction |
| with thiol | compounds | with thiol groups) |
| groups | | |
| Effects on | Halogens | Inhibition of DNA synthesis |
| DNA | Hydrogen | DNA strand breakage |
| | peroxide, | |
| | silver ions | |
| Oxidizing | Halogens | Oxidation of thiol groups to disulfides, |
| agents | | or disulfoxides |
| | Peroxygens | Hydrogen peroxide: activity due to from |
| | | formation of free hydroxy radicals |
| | | (—OH), which oxidize thiol |
| | | groups in enzymes and proteins; |
| | | PAA: disruption of thiol groups in |
| | | proteins and enzymes |

“Biocide” is a general term describing a chemical agent, usually broad spectrum, that inactivates microorganisms. Because biocides range in antimicrobial activity, other terms are more specific, including “-static,” referring to agents which inhibit growth (e.g., bacteriostatic, fungistatic, and sporistatic) and “-cidal,” referring to agents which kill the target organism (e.g., sporicidal, virucidal, and bactericidal). Disinfectants are generally products or biocides that are used on inanimate objects or surfaces. Disinfectants can be sporostatic but are not necessarily sporicidal. Sterilization refers to a physical or chemical process that completely destroys or removes all microbial life, including spores.

Different types of microorganisms vary in their response to decontaminants and disinfectants, as shown below, in descending order.
Prions*
(CJD, BSE)
Coccidia
(Cryptosporidium)
Spores
(Bacillus, C. Difficile)
Mycobacteria
(M. tuberculosis, M. avium)
Cysts
(Giardia)
Small non-enveloped viruses
(Polio Virus)
Trophozoites
(Acanthamoeba)
Gram-negative bacteria (non-sporulating)
(Pseudomonas, Providencia)
Fungi
(Candida, Aspergillus)
Large non-enveloped viruses
(Enteroviruses, Adenovirus)
Gram-positive bacteria
(S. aureus, Enterococcus)
Lipid enveloped viruses
(HIV, HBV)
   The asterisk indicates that the conclusions relating to prions are not yet universally agreed upon.

Organophosphate compounds and other chemical toxants generally have low solubilities in water. Conversely, the redox reagents that can be used to neutralize toxants (e.g., most reactive oxygen species and their dry sources) have very low solubilities in organic solvents. Previous decontamination solutions have been unable to dissolve the two different types of compounds extensively. Current decontamination solutions developed for military use are incapable of dissolving or hydrolyzing significant amounts of organophosphates, nitrogen mustards, or sulfur mustards, and these current decontamination solutions freeze at approximately 32° F. Moreover, prior to the present invention, single decontamination chemical solutions have been unable to decontaminate both chemical and biological toxants using the same formulation.

A decontamination formulation is usually a solution, which refers to a homogeneous mixture composed of two or more substances. In such a mixture, a solute is dissolved in another substance, known as a solvent. The term “solvent” refers to a liquid or gas that dissolves a solid, liquid, or gaseous solute, resulting in a solution. The most common solvent is water. Most other commonly-used solvents are organic (carbon-containing) chemicals. These solvents typically have high melting points, low boiling points, evaporate easily, and have limited if any solubility in water. A typical decontamination formulation is a mixture of two or more substances in a liquid solution, one of which can be an oxidizing agent. In such a mixture, the oxidizing agent is the solute and is dissolved in the solvent. To decontaminate a toxant, solvent-based, decontamination formulations must dissolve and then oxidize, hydrolyze, or otherwise neutralize CW or BW agents, reducing them to non-toxic chemical by-products.

Reactions involved in detoxification of chemical agents are typically hydrolyzing and oxidizing reactions involving reagents which convert the toxant molecules to harmless by-products. Decontamination of biological agents is more complex and is focused on reducing or eliminating the abilities of bacteria, bacterial spores, or viruses to infect a host organism.

Various chemical reactions have been used to decontaminate chemical and biological warfare agents. The chemistries most commonly used in previous decontaminant formulations have relied upon the use of either hypochlorite ion, ClO⁻ (which was usually derived from sodium or calcium hypochlorite (NaClO and Ca(ClO)₂)) or a hydroxyl radical (OH) (derived from hydrogen peroxide or its dry source, sodium peroxide (Na₂O₂)). Sodium peroxide is hydrolyzed by water to form sodium hydroxide and hydrogen peroxide:

Na₂O₂+2 H₂O→2 NaOH+H₂O₂

Further, dry hydrogen peroxide sources (e.g., sodium percarbonate or urea peroxide, also known as carbamide peroxide) can be dissolved in water. Due to their low stability, hypochlorites are also very strong oxidizing agents. The rate of hydrolysis of CWs by hydrogen peroxide or sodium hypochlorite, and the nature of the products formed, depends primarily on the solubility of the agent in water, the pH of the solution, and the relative concentrations of chemical toxant to hypochlorite ions or hydroxyl radicals in the solution.

Other oxidative methods have also been applied as decontaminants of mustard and VX agents (Yang, 1995). An early oxidant used for decontamination was potassium permanganate. More recently, a mixture of KHSO₅, KHSO₄, and K₂Sa₄ was developed as an oxidant for CW decontamination. Several peroxygen compounds have also been shown to oxidize chemical agents (e.g., perborate, peracetic acid, m-chloroperoxybenzoic acid, magnesium monoperoxyphthalate, and benzoyl peroxide). More recently, hydroperoxycarbonate anions produced by the reaction of bicarbonate ions with hydrogen peroxide have been shown to effectively oxidize mustard and VX agents. Polyoxymetalates are being developed as room temperature catalysts for oxidation of chemical agents, but the reaction rates of these compounds have been reported to be slow at this stage of development. As a general rule, oxidation of organophosphate and mustard agents in decontaminants that are predominantly aqueous solutions of oxidizers have been slow and limited.

Various alternative solutions and formulations that have been used for chemical warfare decontamination include supertropical bleach; a non-aqueous liquid composed of 70% diethylenetriamine, 28% ethylene glycol monomethyl ether, and 2% sodium hydroxide, referred to as “Decontamination Solution Number 2” (or DS2); and a mixture consisting of 76% water, 15% tetrachloroethylene, 8% calcium hypochlorite, and 1% anionic surfactant mix.

There are additional compositions that can be used for the decontamination of personnel in the event of a CW agent attack, primarily used by the U.S. military and are, in general, not utilized in the civilian community. One such formulation is the M258 skin kit, which consists of two packets: Packet I contains a towelette prewetted with phenol, ethanol, sodium hydroxide, ammonia, and water and Packet II contains a towelette impregnated with chloramine-B and a sealed glass ampoule filled with zinc chloride solution. The ampoule in packet II is broken and the towelette is wetted with the solution immediately prior to use.

Another military formulation is the M291 kit, which is a solid sorbent system (Yang, 1995). The kit is used to wipe liquid agent from the skin and is composed of non-woven fiber pads filled with a resin mixture. The resin is made of a sorptive material based on styrene/divinylbenzene and a high surface area carbonized macroreticular styrene/divinylbenzene resin, cation-exchange sites (sulfonic acid groups), and anion-exchange sites (tetraalkylammonium hydroxide groups). The sorptive resin can absorb liquid agents and the reactive resins are intended to promote hydrolysis of the reactions.

Most formulations for the decontamination of BW agents used by both military and civilian agencies contain a hypochlorite anion (i.e., bleach or a chlorine-based solution). Solutions containing concentrations of 5% or more bleach have been shown to kill spores (Sapripanti and Bonifacino, 1996). A variety of other hypochlorite solutions have been developed for decontamination of BW agents including 2-6% aqueous sodium hypochlorite solution (household bleach); a 7% aqueous slurry or solid calcium hypochlorite (HTH); 7 to 70 percent aqueous slurries of calcium hypochlorite and calcium oxide (supertropical bleach, STB); a solid mixture of calcium hypochlorite and magnesium oxide, a 0.5% aqueous calcium hypochlorite buffered with sodium dihydrogen phosphate and detergent, and a 0.5% aqueous calcium hypochlorite buffered with sodium. Although all of these solutions are capable of killing spores, each is also highly corrosive to equipment and toxic to personnel.

There are several mechanisms generally recognized for spore (BW) kill. These mechanisms can operate individually or simultaneously. In one mechanism, the dissolution or chemical disruption of the outer spore coat can allow penetration of oxidants into the interior of the spore. Several studies (King and Gould, 1969; Gould et al., 1970) suggest that the S—S (disulfide) rich spore coat protein forms a structure which successfully masks oxidant-reactive sites. Reagents that disrupt hydrogen and S—S bonds increase the sensitivity of spores to oxidants. Additionally, certain surfactants can increase the wetting potential of the spore coat to such an extent as to allow greater penetration of oxidants into the interior of the spore.

Although spores are highly resistant to many common physical and chemical agents, a few antibacterial agents are also sporicidal. However, many powerful bactericides may only be inhibitory to spore germination or outgrowth (i.e., sporistatic), rather than sporicidal. Examples of sporicidal reagents include, but are not limited to, glutaraldehyde, formaldehyde, iodine and chlorine oxyacids compounds, peroxy acids, and ethylene oxide. In general, all of these sporicidal compounds are considered to be toxic in and of themselves, so they do not present a widely useful solution to combat biological warfare terrorism.

A well known decontamination agent is DF-200, also known as Sandia Foam. DF-200 will freeze at temperatures below 32° F., and is ineffective below 40° F. DF-200 is known to take in excess of 30 minutes to neutralize a significant amount of a contaminating substance. Also, DF-200 cannot be used as an aerosol decontaminant, and is not effective against mustards and VX in standard decontaminant tests. Although once identified as the decontaminant of choice by the U.S. Army, in June, 2008, DF-200 was abandoned as a decontaminant. Other well-known decontamination agents for chemical and biological warfare agents include DeconGreen, GD-5/CASCAD, vaporous hydrogen peroxide (VHP), and titanium oxide (TiO₂).

The compounds that have been developed for use in detoxification of CW and BW agents have been deployed in a variety of ways (e.g., liquids, foams, fogs and aerosols, or as vapor). Stable aqueous foams have been used in various applications including fire fighting and law enforcement applications (such as prison riot containment). Such foams, however, have typically been made using anionic surfactants and anionic or non-ionic polymers. These foams, unfortunately, have not been effective in the chemical decomposition and neutralization of most chemical and biological weapons (CBW) agents. They did not have the necessary chemical capabilities to decompose or alter CW agents, nor are they effective in killing or neutralizing the bacteria, viruses and spores associated with some of the more prevalent BW agents.

Gas phase and fogging reagents could be attractive for decontamination, but only if an environmentally acceptable gas or fog can be identified. The advantage of gas or aerosol fog decontaminants is their penetrating capability, which makes them a desirable complement to the other decontamination techniques. Ozone, chlorine dioxide, ethylene oxide, and paraformaldehyde have all been investigated for decontamination applications. These are all known to be effective against biological agents. However, while ozone is an attractive decontaminant, experiments have shown that it is not effective towards GD and VX ozone leads to the formation of toxic products via P—O bond cleavage (Hovanic, 1998).

In addition to being rapidly effective against toxants, a practical decontaminant must be deployable if it is to be used in the field. It must be readily and safely transportable, easy to use even at extreme temperatures (i.e., below 32° F. and above 100° F.), and have a small logistical footprint. A deployable decontaminant should also be effective at low ratios of decontaminant/toxant, be easy to clean up, be environmentally friendly, non toxic, non-flammable, provide excellent material compatibility, and be biodegradable. Finally, a deployable decontaminant should be easy to apply either by direct application or as an aerosol spray.

The present invention addresses, among other advantages, the need for a fully integrated decontamination system that (i) dissolves or solubilizes threat loads of both chemical and biological toxants on surfaces or in aerosol clouds; (ii) provides sufficient concentrations of effective oxidizers to reduce all toxants to safe by-products; (iii) comprises solutions that do not freeze or boil over the temperature range −25° F.≦T≧+125° F., are fully deployable, and which rapidly reduce chemical or biological toxants to aerosol concentrations or surface densities significantly less than the levels established as safe for otherwise unprotected humans.

### BRIEF DESCRIPTION OF THE INVENTION

The invention relates to organic/aqueous liquid solutions useful for neutralizing amphipathic chemical toxants, including but not limited to organophosphates, sulfur mustards and toxic industrial compounds, and also, biopathogens, using a single decontamination solution. The invention reduces, or neutralizes, toxants to harmless biodegradable by-products via perhydrolysis. The invention also includes methods of activating components of the organic/aqueous mixtures to provide reactive oxygen species that are perhydrolyzed to generate the oxidizers, which reduce the toxants to harmless biodegradable by-products. Furthermore, the invention relates to the organic/aqueous liquids useful for neutralizing or killing biological toxants. The term “neutralization” refers to mitigation, detoxification, hydrolysis, reduction to harmless by-products, decontamination, denaturization, or other destruction of toxants that will meet, and if possible, exceed the United States government and military guidelines for decontamination of chemical and biological warfare agents.

Certain embodiments of the present invention comprise systems and methods for enhancing nucleophilic substitutions to of toxants, including, but not limited to, organophosphate esters, including pesticides and nerve agents; blister (also known as sulfur and nitrogen mustard) agents; bacteria and bacterial spores; and viruses.

In other aspects, the organic/aqueous solutions of the invention can be used to create CBW decontaminant formulations by dissolving a reactive oxygen species or its dry source in sufficient amounts to perhydrolyze the amount of a toxant that has been dissolved. The reactive oxygen species can be dissolved in its reactive state. Alternatively, the reactive oxygen species can be inactive when dissolved, or it can be part of another compound when dissolved. It is one aspect of the present invention that the inactive reactive oxygen species can be chemically generated when mixed with other chemicals (“activators”) prior to use of the decontaminant to neutralize toxants. Such activators can also be dry or they can be liquid. Other embodiments include, but are not limited to, surface active components, including but not limited to conventional surfactants and block co-polymers in organic/aqueous mixtures. Surface active components are useful as co-solutes to increase the solubility of the activating components in the organic/aqueous liquid mixtures. Thus another aspect of the present invention are methods of increasing the solubility of the activating components in the organic/aqueous liquid mixtures. Surface active components also reduce the surface tension of the decontaminant formulations, enabling their use in aerosol and fogging applications. Reducing the surface tension of the formulation by use of block co-polymers as surface active agents also makes it possible to aerosolize the decontaminant as a non-Newtonian fluid, which has low viscosity under dynamic shear and forms microemulsions.

As shown below, each molecule of the reactive oxygen species tetraacetylethylenediamine (TAED) is perhydrolyzed at the appropriate pH by activators such as hydroperoxide anions to generate 2 moles of peroxyacetic acid, which, in turn, form percarboxylate anions and/or singlet oxygen. These molecules can react with a threat load of toxant, and neutralize/remove the toxant without the production of toxic by-products.

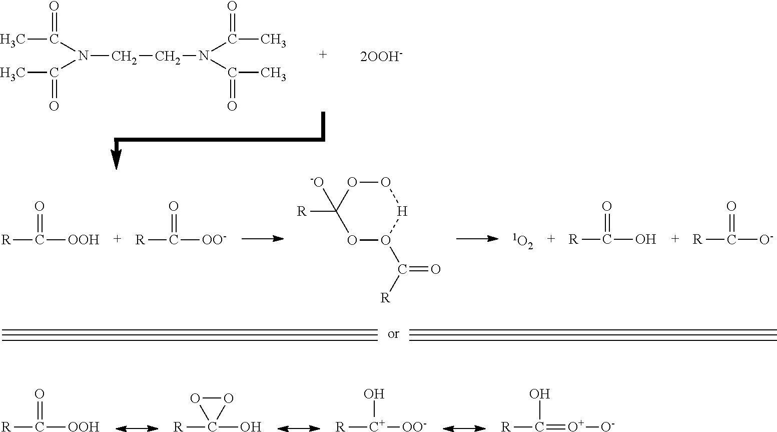

### Generation of Peroxyacetic acid and its Oxidizers from TAED

In the present invention, hydroperoxide anions, which perhydrolzye the reactive oxygen species, are produced by the chain propagation reaction to generate percarboxylate anions and singlet oxygens:

H₂O₂+OH. [symbol] HO₂.+H₂O

HO₂. [symbol] H⁺+O₂.⁻

HO₂.+O₂.⁻ [symbol] HO₂⁻+O₂

The desired percarboxylate anions and singlet oxygens cannot be generated from either hydrogen peroxide alone or from sodium hypochlorite alone. In the present invention, these are generated through the generation of peroxyacetic acid and the subsequent generation of percarboxylate and singlet oxygen oxidizers, as a result of the perhydrolysis of TAED. Moreover, these compounds react with organophosphates, mustards, bacteria, spores, and viruses via different reaction pathways and mechanisms from those generated by activation of hydrogen peroxide or sodium hypochlorite. These different reaction pathways can be exploited to increase the efficacy of the decontamination solutions of the invention and avoid the creation of hazardous by-products.

In one embodiment, the invention relates to a system for decontaminating chemical and biological agents comprising a polar organic amphipathic solvent; an activator; and a reactive oxygen species; whereupon mixing, the amounts of the polar organic amphipathic solvent, the activator and the reactive oxygen species are sufficient to maintain a pH of less than or equal to about 8.5 and to produce an amount of singlet oxygen molecules or percarboxylate anions to decontaminate a threat load of toxant. The pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

In one embodiment, the decontaminant formulation, or the component mixtures from which it is prepared, can comprise a dry activator and at least one liquid component containing one or more reactive oxygen species or their dry sources.

In another embodiment, the decontaminant formulation, or the component mixtures from which it is prepared, can comprise a liquid activator and at least one liquid component containing one or more reactive oxygen species or their dry sources. In one embodiment, the activator can be a known activator of the reactive oxygen species, including hydrogen peroxide or its dry source, which are known activators of reactive oxygen species such as tetraacetylethylenediamine (TAED), sodium nonanoyloxybenzenesulfonate (NOBS), or any of the related fatty acid type anionic surfactant activators. This includes, but is not limited to, decanoic acid, 2-[[(4-sulfophenoxy)carbonyl]oxy]ethyl ester (DECOBS).

In yet another embodiment, the chemical activator, in a liquid or dry form, can be any of a number of peroxide or persulfate sources which activate organic peroxyacids, such as peroxycarboxylic acids or compounds which generate and then activate peroxycarboxylic acids. This includes, but is not limited to, peroxyacetic acid, peroxypropanoic acid, or peroxyoctanoic acid. These compounds can be formed from liquid or dry sources, including, but not limited to, tetraacetylethylenediamine (TAED) or tetraacetylymethylenediamine (TAMD,) peracids chosen from the imidoperacids with the general structure:

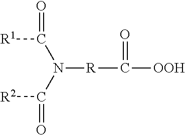

The activator can also be a diperacid represented by the general structure HO₃—(CH₂)ₚ—CO₃H wherein p is any number between 2 and 10, such as diperoxydodecanedioic acid (DPDDA), any peroxyacid sources selected from compounds having the general formula

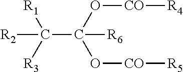

or any other such peroxygen acid precursors. TAED has the chemical formula (CH₃C(O))₂NCH₂CH₂N(C(O)CH₃)₂, and can be perhydrolyzed by:
hydrogen peroxide from any source, including but not limited to persalts such as sodium perborate, sodium percarbonate, calcium peroxide, magnesium peroxide, zinc peroxide, thiourea dioxide, urea hydrogen peroxide (carbamide peroxide, urea peroxide, and percarbamide), or
persulfate sources such as potassium monopersulfate.

In a preferred embodiment of the present invention, chemical activation entails reaction of hydrogen peroxide from any sources with TAED to release two moles of peroxyacetic acid (also known as “peracetic acid”) per mole of TAED. Hydrogen peroxide is an inefficient reactive oxygen species in a decontaminant by itself, whereas the peroxycarboxylic acids are fast-acting oxidizing agents with a high oxidation potential:

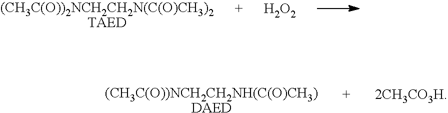

The at least one liquid activator can be selected from peroxides, hydroxyl radicals, hydroxyl ions, hydroperoxide anions, superoxides, persalts, persulfates, and peroxyacetic acid. In one embodiment, the decontamination mixture can be prepared using two liquid activators. In another embodiment, a first liquid activator comprises hydrogen peroxide.

In yet another embodiment, the decontamination mixture can include a surface active compound that is a block co-polymer, which will impart better aerosolization capabilities to the decontamination solution. The block co-polymer may be an ethylene oxide and propylene oxide di- or tri-block co-polymer. More specifically, the block co-polymer may be an ethylene oxide and propylene oxide co-polymer that terminates in primary hydroxyl groups.

The invention also relates to a system for decontaminating chemical and biological agents comprising at least a water-soluble polar organic amphipathic solvent; an activator that provides a buffering system to establish and maintain a pH of about 8.0 to about 8.5; and a reactive oxygen species (ROS). Upon mixing these three components with water, a single-phase, aqueous organic solution is formed. The solution produces and maintains a sufficient amount of singlet oxygen molecules and/or percarboxylate anions to decontaminate a threat load of toxant. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

The invention also relates to a method of decontaminating, or neutralizing, a chemical or biological toxant, the method comprising mixing a water-soluble polar organic amphipathic solvent, an activator that provides a buffering system to establish and maintain a pH of about 8.0 to about 8.5, and a reactive oxygen species with water to form a single-phase aqueous, which can be transparent, decontamination mixture; and physically associating the decontamination mixture with the toxant, wherein the solution produces and maintains a sufficient amount of singlet oxygen molecules or percarboxylate anions, thereby decontaminating a threat load of toxant. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

The invention also relates to a method of decontaminating, or neutralizing, a chemical or biological toxant, further comprising testing for the presence of the toxant; and repeating the steps of mixing the water-soluble polar organic amphipathic solvent, the activator that provides a buffering system to establish and maintain a pH of about 8.0 to about 8.5, and the reactive oxygen species with water to form a single-phase aqueous decontamination solution; and physically associating the decontamination solution with the toxant until the level of the toxant is reduced by at least 99.4%, wherein the solution produces and maintains a sufficient amount of singlet oxygen molecules or percarboxylate anions, thereby decontaminating a threat load of toxant. The decontamination solution can be transparent.

The invention further relates to a method of decontaminating a toxant, which method comprises providing an organic/aqueous solution comprising at least two polar amphipathic solvents which can be water-soluble and selected from the groups consisting of the solvents identified above and in Table 2 below, wherein the volume fraction of water in the solution ranges from about 25% to about 75%, and the final pH of the solution is less than or equal to about 8.5; providing at least one chemical activator that provides a buffering system to establish and maintain a pH of about 8.0 to about 8.5; providing at least one reactive oxygen species; mixing the solution, the activator, and at least one reactive oxygen species or its source to form a decontamination mixture; and physically associating the decontamination mixture with the toxant. The decontamination mixture can be physically associated with the toxant by dispersing the decontamination mixtures as an aerosol. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

TABLE 2
The Groups of Polar Amphipathic Solvents used in the Organic/Aqueous Solutions of the Invention

| GROUP | GROUP I | GROUP II |
|---|---|---|
| Property | Polar, aprotic | Polar, protic |
| Organic Solvents | Organic Solvents | Organic Solvents |
| Oxygen content | No | Yes |
| Dipole Moment | Strong | Strong to Weak |
| General Structures | Nitriles: R—C≡N | alcohol: R—OH |
| | Haloalkanes: R—CH₂—X | |
| Example 1 | H₃C—C≡N | H₃C—CH2—OH |
| Name | Acetonitrile | ethanol |
| Example 2 | H₃C—CH₂—CH₂C≡N | CH₃—CH₂—CH₂—CH₂—OH |
| Name | butanenitrile | n-butanol |
| Formula | C₄H₇N | C₄H₁₀OH |
| GROUP HYBRIDS | | 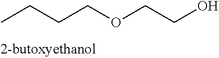 |
| Organic/aqueous | 1 to 99 | 100 to 225 |
| solution number | | |
| GROUP | GROUP III | GROUP IV |
| Property | Polar, aprotic | Polar, polyprotic |
| Organic Solvents | Organic Solvents | Organic Solvents |
| Oxygen content | Oxygen Containing but does not | Polyhydroxyl. Contains many |
| | donate a hydrogen bond. | hydroxyls. Can donate and accept |
| | Can accept a hydrogen bond | many hydrogen bonds |
| Dipole Moment | Strong to Weak | Strong to Weak |
| General Structures | Ketones: R—CO—R′ Ethers: R—O—R′ Aldehydes: R—CO—H | 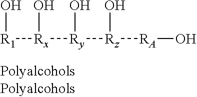 |
| Example 1 Name | H₃C—C(O)H—CH₃ Acetone | 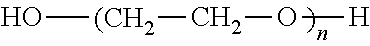 |
| Example 2 Name Formula | 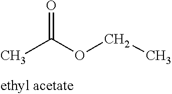 | 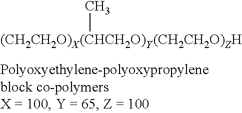 |
| GROUP HYBRIDS | 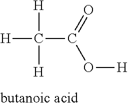 | |
| Organic/aqueous | 225 to 325 | 335 to 550 |
| solution number | | |

As above, the decontamination mixture prepared and used by the method of the invention can be an organic/aqueous solution comprising at least two polar amphipathic solvents which can be water-soluble and selected from the groups consisting of the solvents listed in Table 2. Solvents can include, but are not limited to, nitriles, ketones, aldehydes, amides, furans, alkanols and polyols. The volume fraction of water in the solution ranges from about 25% to about 75%, and the final pH of the solution is less than or equal to about 8.5. Exemplary solvents include the isomers of butanediol and any of the linear or branched-chain alcohols. The linear or branched-chain alcohols can have from 1 to 15 carbons. The solvents can be mixed with activators or percarboxylic acids or their sources prior to use in decontaminating toxants. The activators can be dry activators and liquid activators. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

In one embodiment, the dry activator can be tetraacetylethylenediamine (TAED) or sodium nonanoyloxybenzene-sulfonate (NOBS) or any of the persalts. The at least one activator can be selected from peroxides, hydroxyl radicals, hydroxyl ions, super oxides, or their dry sources. The decontamination mixture can be prepared using at least one liquid activator or one peroxygen source prior to activation for use as a decontaminant. In another embodiment, a first liquid activator can comprise acetic acid and hydrogen peroxide. Alternatively, a second liquid activator comprises a solution of a persalt or a buffering salt such as sodium percarbonate.

In yet another embodiment, the chemical activation of the decontamination mixture can be regulated by using the buffering capacity of the persalt to regulate the pH of the decontaminant during chemical activation. This will maximize the generation of the reactive oxygen species from their sources. For example, sodium percarbonate can be used to buffer pH of the formulation during perhydrolysis of TAED to generate peroxyacetic acid.

The term “buffer capacity” refers to the amount of an acid or base that can be added to a volume of a buffer solution before its pH changes significantly. Water is subject to self-ionization but has no buffer capacity so that generation of peroxycarboxylic acid in an unbuffered formulation rapidly ceases after the initial perhydrolysis. The primary oxidizer can be the hydronium ion, HOO—, which is produced when hydrogen peroxide is used as the reactive oxygen species in alkaline formulations. Addition of NaOH alone to an unbuffered formulation causes off gassing of the peroxide activator, again stopping the perhydrolysis prematurely.

In the present invention, the buffer capacity is a quantitative measure of the resistance of the decontaminant solution to pH change on addition of hydroxide or H+ ions and can be defined as follows:

$$buffer capacity = \frac{d n}{d ( pH )}$$

where dn is a small amount of added base and d(pH) is the resulting infinitesimal change in pH. With this definition the buffer capacity can be expressed as:

$$\frac{d n}{d ( pH )} = 2.303 ( \frac{{K}_{w}}{[ {H}^{+} ]} + [ {H}^{+} ] + \frac{{C}_{A} {K}_{a} [ {H}^{+} ]}{{( {K}_{a} + [ {H}^{+} ] )}^{2}} ) ,$$

where K_{w} is the self-ionization constant of water and C_{A} is the analytical concentration of the acid, equal to [HA]+[A⁻]. The term K_{w}/[H⁺] becomes significant at pH greater than about 11.5 and the second term becomes significant at pH less than about 2. Both these terms are properties of water and are independent of the weak acid. Considering the third term, it follows that:
Buffer capacity of a weak acid reaches its maximum value when pH=pKₐ.
At pH=pKₐ±1 the buffer capacity falls to 33% of the maximum value. This is the approximate range within which buffering by a weak acid is effective. Note: at pH=pKₐ−1, the Henderson-Hasselbalch equation shows that the ratio [HA]:[A⁻] is 10:1.
Buffer capacity is directly proportional to the analytical concentration of the acid.

In the preferred embodiment of the present invention, the pKₐ, buffer and the buffer capacity are selected from the acids and conjugate bases which have a pKₐ of 8.5±1 and which have the buffer capacity to enable generation of sufficient perhydrolysis of TAED or other peroxycarboxylic acid sources to produce sufficient oxidizers to neutralize a full threat load of chemical agent. In yet another novel discovery of the present invention, as shown in FIG. 3, appropriate selection of the pKₐ and the buffer capacity enables regulation of perhydrolysis over time, so that the activated “pot-life” of the decontaminant can be prescribed. The term “pot-life” refers to the time period during which the formulation is optimally active.

In yet another embodiment, the decontamination mixture can comprise a block co-polymer, which will impart better solubilization of the reactive oxygen species or its source and improve the aerosolization capabilities of the decontamination solution. The block co-polymer can be an ethylene oxide and propylene oxide co-polymer. More specifically, the block co-polymer can be an ethylene oxide and propylene oxide co-polymer that terminates in primary hydroxyl groups.

In another embodiment, the method of the invention may be used to decontaminate various toxants. The toxant may be, for example, a phosphoric acid ester, a sulfur mustard, bacteria, bacterial spores, or viruses. The decontamination may be carried out via chemical modification and perhydrolysis of the toxant, on a surface, in an aerosol suspension, or a combination thereof. If applied as an aerosol, the mixture can be dispersed through a nozzle as a microemulsion. In general, the total decontaminant to toxant volume ratio can range from about 200 to about 0.1.

The temperature at which decontamination using the present invention may be carried out has a wide range. For example, the decontamination can be conducted at a temperature of between about −25° F. and about 125° F. Alternatively, the decontamination can be conducted at a temperature of between about −35° F. and about 140° F.

The method can further comprise testing for the presence of the toxant; and repeating the steps of providing the solution, activator, and at least one reactive oxygen species or source thereof, mixing the solution, activator, and at least one reactive oxygen species, and physically associating the decontamination mixture with the toxant until the level of the toxant present is reduced in concentration by at least two (2) logs from the initial threat load.

In another embodiment, the decontamination solution may also comprise a surfactant, or block co-polymer, and/or a fluorescent dye.

Another embodiment of the invention relates to a kit or system comprising a solution comprising at least two polar water-soluble organic amphipathic solvents selected from Groups I through IV (See Table 2), and including but not limited to, nitriles, ketones, aldehydes, amides, furans, alkanols and polyols, wherein the volume fraction of water in the solution ranges from about 25% to about 80% or from about 25% to about 75%, and the pH of the solution is less than or equal to about 8.5; an activator; and at least one liquid reactive oxygen species or source thereof. The polar amphipathic solvents can be water-soluble and can be any of the Groups I through IV solvents identified in Table 2, alone or in mixtures, such as the isomers of butanediol (Group IV) or any of the Group II solvents, such as ethanol or hexanol. The activator can be, but is not limited to, hydrogen peroxide or any peroxide or persulfate sources, a peroxycarboxylic acid or any peroxy acid or source thereof. The at least one reactive oxygen species can be a peroxide or a peroxycarboxylic acid, such as peroxyacetic acid or any source thereof. This includes, but is not limited to, the persalts and TAED. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

In one embodiment, the kit or system may comprise an organic/aqueous solution including at least two polar amphipathic solvents, which can be water-soluble and selected from Groups I through IV (See Table 2), and at least one activator. One of the activator can be hydrogen peroxide or a source thereof, and a second activator can be any buffering salt. The system may further comprise a surface active agent and/or a fluorescent dye.

The kit or system may further be distinguished by a formulation number such as those given at the bottom of Table 2 above. This number identifies the composition of the organic/aqueous solution that is used in its creation.

The kit or system may further comprise a container for mixing the organic/aqueous solution comprising at least two polar amphipathic solvents selected from Groups I through IV (See Table 2), at least one liquid or dry activator and at least one liquid or dry reactive oxygen species or source thereof together into a decontamination mixture; and means for physically associating the decontamination mixture with the toxant. The means for physically associating the decontamination mixture with the toxant comprises at least an aerosolization nozzle, but may include a mixing chamber.

### BRIEF DESCRIPTION OF THE DRAWINGS

The accompanying drawings, which are incorporated in and constitute a part of this specification, illustrate an implementation of the present invention, and together with the description, serve to explain the advantages and principles of the invention. In the drawings:

FIG. 1 depicts the activation of an exemplary solution of the invention.

FIG. 2 depicts a flow chart disclosing the activation and use of the invention.

FIG. 3 depicts the rate of perhydrolysis of TAED by H₂O₂ in unbuffered water as a function of pH.

FIG. 4 depicts the pKₐ of acetic acid in a dioxane/water solution.

FIGS. 5A-5C depict graphs relating to melting and boiling points of various compounds. FIG. 5A shows a comparison between the boiling points of water and other small molecules with similar molecular structures. There is a steady increase in boiling point in the series CH₄, GeH₄, SiH₄, and SnH₄. The boiling point of H₂O, however, is anomalously large because of the strong hydrogen bonds between water molecules. FIG. 5B shows a comparison of melting points. FIG. 5C relates to melting point depression behavior for various solutions.

FIGS. 6A-6B depict examples of the physical behavior of pseudoplastic non-Newtonian fluids demonstrating the property known as “shear-thinning.” In FIG. 6A, as the force increases, the shear also increases, whereas in FIG. 6B, as the shear increases, the viscosity or resistance to flow decreases.

FIG. 7 depicts a comparison of the effects of shear on the viscosity of fluids.

FIG. 8A shows the pseudoplastic behavior of carboxymethyl cellulose (CMC); FIG. 8B shows a relative viscosity profile of various CMCs.

FIG. 9 shows that viscoelastic behavior can differ with temperature.

FIG. 10 depicts a schematic of how droplet size is determined.

FIG. 11 shows different nozzles which can be used to aerosolize non-Newtonian fluids.

FIG. 12 depicts a standard curve for the detection by absorbance of light at 257 nm by the organophosphate ester diphenyl phosphorochloridate (DPCP), a G agent chemical simulant.

FIG. 13 depicts a quaternary configuration of a kit of the invention.

FIG. 14 depicts a binary configuration of a kit of the invention.

FIG. 15 depicts the flow of a viscous fluid through the walls of a pipe.

FIG. 16 is an illustration of a viscosity model for viscous fluids.

FIG. 17 depicts the results of the hydrolysis of an organophosphate ester by a reactive oxygen species in an organic/aqueous solution.

### DETAILED DESCRIPTION OF THE INVENTION

Embodiments of the present invention will now be described in detail with reference to the drawings as illustrative examples so as to enable those skilled in the art to practice the invention. Notably, the figures and examples are not meant to limit the scope of the present invention to any single embodiment; other embodiments are possible by way of interchange of some or all of the described or illustrated elements. Where certain elements of these embodiments can be partially or fully implemented using known components, only those portions of such known components that are necessary for understanding the present invention will be described, and detailed descriptions of other portions of such known components will generally be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Further, the present invention encompasses present and future known equivalents to the components referred to herein by way of illustration.

The invention is based upon several axioms of the inventor's general model of chemical and/or biological decontamination. First, the decontamination formulation should be able to sufficiently dissolve full chemical threat loads (See Table 4, below) in an isotropic homogenous solution. Second, the decontamination formulations should preferably be able to dissolve sufficient amounts of one or more reactive oxygen species or their sources in the isotropic solution, where the reactive oxygen species can react with the dissolved toxant to reduce a chemical of biological threat load to a substantially harmless level. Third, the volume ratio for neutralizing threat loads generally determines the reaction rate and logistical footprint and should preferably be less than 50:1 decontaminant to toxant. Fourth, the decontaminant should be a low viscosity fluid, at least under dynamic shear, which is both fluid and effective against a chemical or biological agent over a temperature range from about −25° F. to about 125° F. Fifth, the decontaminant should preferably remain in contact with the toxant long enough to substantially complete perhydrolysis by, for example, having a sufficiently high viscosity on a surface. Sixth, an effective decontamination solution for chemical agents should preferably be able to reduce chemical agents to one or more harmless by-products. Finally, a deployable chemical and biological decontaminant should preferably meet and, if possible, exceed CFR49, DOT and DoD transport requirements, NIOSH and EPA ESHO requirements, DOD material compatibility requirements, and all needs for ease of transport and storage, ease and safety of use, ease of clean-up, safe disposal, material compatibility and biodegradability.

In addition, a deployable chemical and biological decontaminant should meet, and if possible, exceed the United States Government and Military guidelines for decontamination of chemical and biological warfare agents. See Tables 3 and 4 below. Specifically, the Joint Program Executive Office (JPEO) for chemical and biological defense has listed the following guidelines for VX, GD, and HD (nerve agents) and biological agents, such as Anthrax.

TABLE 3
Reduction of Toxant

| TOXANT | VX | GD | HD | Anthrax |
|---|---|---|---|---|
| Percent Reduction | 99.9995% | 99.95% | 99.4% | 99.9999% |
| Log Reduction | 5.5 log | 3.5 log | 3.4 log | 6 log |

TABLE 4
Reduction of Toxant
Contact Exposure Level Goals (mg/m²):
Starting Threat Load of 10 g/m² (at −25° F. ≦ T ≦ 125° F.)
(Surface Decontamination)

| Level | Nerve G | Nerve V | Blister H |
|---|---|---|---|
| DTRA/JPEO - | <1.7 | <0.04 | <3.0 |
| Threshold | | | |
| DTRA/JPEO - | 0 | 0 | 0 |
| Objective | | | |

Vapor Level Goals (mg/m³):
Starting Threat Load of 10 g/m³ (at −25° F. ≦ T ≦ 125° F.
(Aerosol Decontamintation)

| Level | Nerve G | Nerve V | Blister H |
|---|---|---|---|
| DTRA/JPEO - | <0.000870 | <0.000036 | <0.0058 |
| Threshold | | | |
| DTRA/JPEO - | <0.00020 | <0.000024 | <0.0030 |
| Objective | | | |

Residual Levels of Biological Agents:
Starting Threat Load >10⁸ Spores/m² (at −25° F. ≦ T ≦ 125° F.)

| Level | Bacterial Endospores | Vegetative Bacteria | Viruses |
|---|---|---|---|
| DTRA/JPEO - | <100 | <10 | <10 |
| Threshold | | | |
| DTRA/JPEO - | 0 | 0 | 0 |
| Objective | | | |

Chemical/Biological Requirements (Aerosol Cloud)
(at −25° F. ≦ T ≦ 125° F.)

| Level | Chemical (VX, G, others) | Bacteria/Viruses |
|---|---|---|
| DARPA - | 4 log | 4 log |
| Objective | | |

In certain embodiments of the invention, an organic/aqueous solution comprises at least one polar amphipathic organic solvent, which can be water-soluble and selected from one or more of the groups consisting of solvents given in Table 2, including: Group I solvents, which comprise nitriles or other polar aprotic solvents that contain no oxygen; Group II solvents, which comprise alkanols or other polar, monoprotic solvents that contain one —OH moiety; Group III solvents, which comprise aldehydes, ketones, ethers, or other polar, aprotic solvents that contain oxygen but cannot donate a proton; and Group IV polyprotic solvents and solutes, which comprise polyols, and/or solvents designated hybrids because they share properties of several groups, such as 2-butoxyethanol.

The volume fraction of water in such organic/aqueous solutions can preferably range from about 25% to about 80% or from about 25% to about 75% and the final solution pH can preferably have a value less than or equal to about 8.5. In certain embodiments of the invention organic/aqueous solutions further comprise at least one reactive oxygen species or at least one oxidizing agent. Suitable reactive oxygen species include, but are not limited to, peroxides and peroxycarboxylic acids; suitable oxidizers include but are not limited to hydroxyl radicals, hydroxyl ions, hydronium ions, hydroperoxide anions, superoxides, ozone, hydroperoxide anions, peroxyacid anions, such as peroxyacetic anion or peroxyoctanoic anion, and/or singlet oxygen. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

The method of the invention contains a series of steps. These steps include providing a solution comprising least one water-soluble polar organic amphipathic solvent providing at least one dry or liquid activator and a reactive oxygen species; mixing the solution, the dry activator, the one liquid activator, and the reactive oxygen species to form a decontamination mixture; and physically associating the decontamination mixture with the toxant. Each solvent can be a polar aprotic solvent, a polar-protic solvent, or combinations thereof. More specifically, the polar organic amphipathic solvent can be a nitrile, a ketone, an aldehyde, a carboxylic acid, an amide, a furan, an alkanol, a polyol, or combinations thereof.

The volume fraction of water in the solution can range from about 25% to about 75%, and the pH of the solution is less than or equal to about 8.5. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5. In one embodiment, the invention relates to a system for decontaminating chemical and biological agents comprising a polar organic amphipathic solvent; an activator; and a reactive oxygen species; whereupon mixing, the amounts of the water-soluble polar organic amphipathic solvent, the activator and the reactive oxygen species are sufficient to maintain a pH of less than or equal to about 8.5 and to produce an amount of singlet oxygen molecules or percarboxylate anions to decontaminate a threat load of toxant. Alternatively, the pH can be maintained at less than or equal to about 8.0 The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

As used herein, the term “nitrile” refers to any molecule or organic compound or solvent that contains a —C≡N functional group in which the carbon atom and the nitrogen atom are triple bonded together. Examples of nitriles include, but are not limited to acetonitrile and rose nitrile. The prefix cyano- is used in chemical nomenclature to indicate the presence of a nitrile group in a molecule. The term “ketone” or “aldehyde” refers to any molecule or organic compound or solvent that contains a —CH_{X}═O functional group in which the carbon atom and the oxygen atom are double bonded together. The term “alkanol” refers to any organic compound or solvent containing a single —OH group in its chemical structure. Examples of alkanols include, but are not limited to, straight chain alcohols, such as methanol, ethanol, propanol, isopropanol, butanol and hexanol. The term “polyol” refers to any organic solvent or solute containing at least two —OH groups in its chemical structure. In polymer chemistry, polyols are compounds with multiple hydroxyl functional groups available for organic reactions.

The term “polyol” includes, but is not limited to: (i) diols (e.g., ethylene glycol, polyethylene glycol, propylene glycol, polypropylene glycol, and any of the isomers of propanediol, butanediol or pentanediol); (ii) triols, which are organic compounds containing three hydroxyl groups (e.g., the trihydric alcohol 1,2,3-propane-triol, CH₂(OH)CH(OH)CH₂(OH), (glycerol); and (iii) polyols, including higher order polyols, which include any organic compound having more than two —OH groups (e.g., polyethylene glycol, polypropylene glycol, and poly(tetramethylene ether) glycol and the sugar polyols.

The main use of polymeric polyols is as reactants to make other polymers. For example, polymeric polyols can be reacted with isocyanates to make polyurethanes, which use consumes most polyether polyols. Common polyether diols are polyethylene glycol, polypropylene glycol, and poly(tetramethylene ether) glycol. The preferred polyols of the present invention are low molecular weight diols and triols based on simple carbon chains, and the polyols known collectively as polyoxyethylene-polyoxypropylene block co-polymers. These compounds have low vapor pressures, high boiling points, low freezing points, high valued flash points, and low viscosities, especially under dynamic shear. These compounds can also readily solubilize both amphipathic organophosphates and sources of the reactive oxygen species and chemical activators.

The solvents used for toxant hydrolysis can be polar, aprotic and/or polar, protic solvents, or a combination thereof, or polar, protic solvents individually. The use of these solvents can promote certain types of nucleophilic attack and thereby enhance the oxidation and hydrolysis or perhydrolysis of phosphate ester and blister agents, as well as of phospholipids, proteins, and DNA or RNA.

In certain embodiments of the invention, hydrolysis or perhydrolysis of toxants can be enhanced by using reactive oxygen species, including, but not limited to hydrogen peroxide and peroxyacetic acid. These two compounds represent entirely different groups of chemical compounds. A peroxide is a compound containing an oxygen-oxygen single bond, while peroxy acids (also known as peroxyacids and peracids) are acids in which an acidic —OH group has been replaced by an —OOH group. Peroxides and peroxy acid have the general structures:

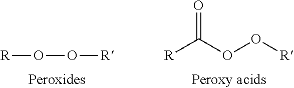

Peroxides tend to decompose easily and can sometimes initiate explosive reactions. Peroxy acids are generally not very stable in solution and decompose to ordinary oxyacids and oxygen. One novel discovery of the present invention is that dry sources of the peroxides, and separately, dry sources of the peroxy acids, such as TAED, are stable when dissolved in the organic/aqueous solutions of the invention, and require activation for maximum efficacy and speed against chemical and biological agents, including, but not limited to, aromatic hydrocarbons, mustards, environmental mutagens, organophosphate pesticides, nerve agents and bacteria.

Hydrogen peroxide decontaminates chemical warfare agents (CWAs) more efficiently in alkaline solutions that generate HOO⁻. In some instances, the alkaline perhydrolysis process is considerably faster than analogous alkaline hydrolysis or neutral oxidation processes. This is attributed to an increased nucleophilicity of HOO— due to the presence of a lone pair of electrons on the oxygen atom adjacent to the nucleophilic centre. This phenomenon is referred to as the ‘α-effect’. Although not fully understood, α-effects are historically considered not to occur in the absence of solvent. However, the observed chemistry of modified vaporous hydrogen peroxide (mVHP) is analogous to the alkaline perhydrolysis chemistry observed in solution. Some of the many difficulties observed using modified vaporous hydrogen peroxide as a CBW decontaminant are: (i) the rapid outgassing of H₂O₂ under alkaline conditions, resulting in a short effective pot life; (ii) the caustic character of the alkaline solutions needed to create mVHP; (iii) mVHP can only be used in enclosed spaces in which heated dry air must be circulated to reduce the relative humidity and avoid condensation of hydrogen peroxide and water during decontamination, and, (iv) the production of toxic by-products produced when mVHP is the primary reactive oxygen species.

Peroxy-oxidizers, including but not limited to, peroxycarboxylic acids such as peroxyacetic acid, and dry sources thereof, can dissolve in the isotropic organic/aqueous solutions of the polar amphipathic organic components of the present inventions. When appropriately activated, these compounds can generate oxidizing agents which (i) overcome the limitations of mVHP; and (ii) accelerate the hydrolysis of phosphate esters, mustards, as well as the phospholipids, proteins and oligonucleotides of bacteria, spores and viruses.

As discussed above, a molecule of the reactive oxygen species tetraacetylethylenediamine (TAED) is perhydrolyzed at the appropriate pH by activators such as hydroperoxide anions, and will generate 2 moles of peroxyacetic acid, which, in turn, form percarboxylate anions and/or singlet oxygen. These oxidizer molecules can react with a threat load of toxant, and neutralize/remove the toxant without the production of toxic by-products.

In the present invention, hydroperoxide anions, which perhydrolzye the reactive oxygen species, are produced by the chain propagation reaction to generate percarboxylate anions and singlet oxygens:

H₂O₂+. [symbol] HO₂.+H₂O

HO₂. [symbol] H⁺+O₂.⁻

HO₂.+O₂.⁻ [symbol] HO₂⁻+O₂

The desired percarboxylate anions and singlet oxygens cannot be generated from either hydrogen peroxide alone and cannot be generated using sodium hypochlorite, but only from the generation of peroxyacetic acid and its oxidizers from TAED. Moreover, these compounds react with organophosphates, mustards, bacteria, spores, and viruses via very different reaction pathways and mechanisms from those generated by activation of hydrogen peroxide or sodium hypochlorite. These different reaction pathways can be exploited to increase the efficacy of the decontamination solutions of the invention and to avoid the creation of hazardous by-products.

In order to be most effective in a decontaminant made from the organic/aqueous solutions of the present invention, the reactive oxygen species should be able to dissolve in sufficient amounts to achieve stoichiometric hydrolysis and/or perhydrolysis of the toxants. The term “reactive oxygen species” (“ROS”) refers to peroxides or peroxyacids, whereas the term “oxidizers” refers to reactive oxygen which may be in the form of hydroxyl radicals (from peroxides) or peroxycarboxylic anions and singlet oxygen (from peroxy acids). As a group, reactive oxygen species include, but are not limited to, hydrogen peroxide, hypochlorite ion, and peroxyacetic acid (PAA). These compounds require some type of activation process during which one or more molecules are split to generate the oxidizing agents. Such oxidizing agents include, but are not limited to, hydroxyl radicals, or peroxyacetic anions and singlet oxygens, which may go on to participate in further chemical reactions with toxants.

The term “radical” or “free radical” refers to a cluster of atoms, one of which contains an unpaired electron in its outermost shell of electrons. The term “hydroxyl” describes a molecule consisting of an oxygen atom and a hydrogen atom joined by a covalent bond. The neutral form is known as a hydroxyl radical and the singly-charged hydroxyl anion is called hydroxide. Hydroxyl radicals are an unstable configuration, and such radicals generally quickly react with other molecules or other radicals to achieve the stable configuration of four pairs of electrons in their outermost shell (or one pair for hydrogen).

Other embodiments of the invention are organic/aqueous solutions comprising one or more reactive oxygen species, further comprising one or more chemical activators to generate the oxidizer(s). When dissolved, the peroxycarboxylic acids require activation to generate the peroxy anions or singlet oxygen atoms which are the actual oxidizers of the toxants. Methods of activation include, but are not limited to: (i) changing the pH of the solution by adding an alkaline base such as sodium hydroxide to the decontamination mixture; (ii) changing the pH by employing buffering systems that both elicit and regulate activation of the perhydrolyzers; (iii) employing catalysts (e.g., NaI, Fe++, transition metals, and lanthanides); (iv) ozonolysis; (v) exposure to ultraviolet light; and/or (vi) use of organic precursor compounds. One such group of organic activator compounds are the persalts which, in the present invention, is used in the organic/aqueous solutions as a dry source of hydrogen peroxide, which under appropriate conditions of pH, can quickly perhydrolyze TAED to generate peroxyacetic acid, as shown above. One novel discovery of the invention is that such organic activators (e.g., sodium persulfate, sodium perborate, sodium percarbonate and/or urea peroxide), can also be used as components of a buffering system to regulate the release of peroxyacetic acid at a prescribed concentration over time. Such a “quasi-steady state equilibrium” effectively lengthens the active pot life of a decontaminant, yet eliminates the outgassing of the peroxide activators, which is another novel aspect of the present invention.

Additional embodiments of the invention comprise organic/aqueous solutions comprising one or more oxidizing agents, an activator to generate the oxidizer, surfactants, and co-polymers, the ability of the decontaminants to remain effective at extreme temperatures, and the use of block co-polymers to create non-Newtonian decontaminants that can be aerosolized as microemulsions and fogs. Yet other embodiments of the invention comprise organic/aqueous solutions comprising one or more reactive oxygen species, a buffering activator to generate the reactive oxygen species and the oxidizer as part of a quasi-steady state equilibrium process, extending the effective life of the decontaminant from minutes to hours and even days.

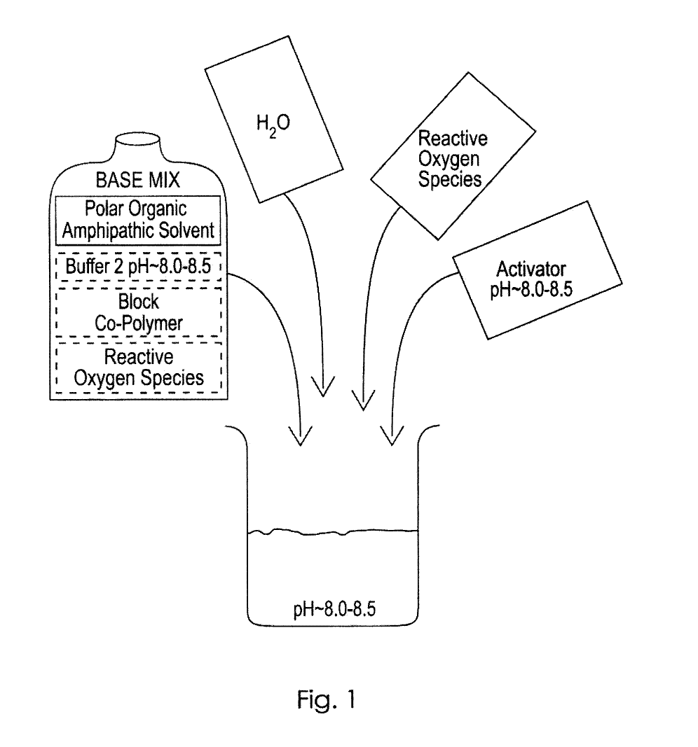

As seen in FIG. 1, the components of the system can be mixed into a container immediately prior to use. The base mix contains the polar organic amphipathic solvent. Other components include at least one reactive oxygen species and at least one activator. The activator can include liquid and/or dry activators, and provides buffering capacity. The base mix can further include a second buffer, a block co-polymer and a reactive oxygen species, depending upon the final configuration of the system.

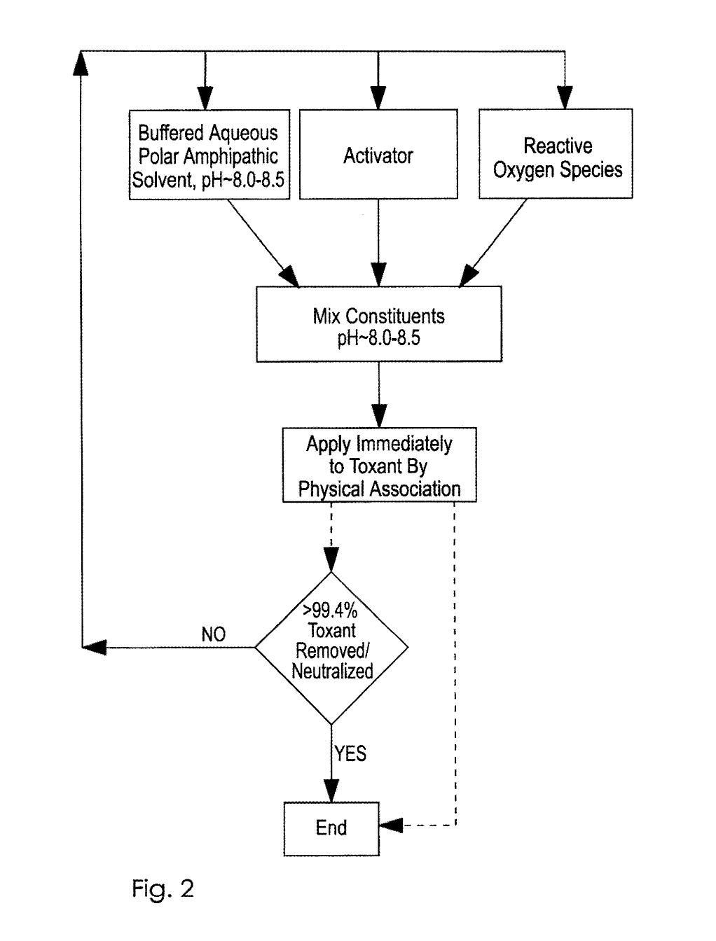

FIG. 2 shows the method of the invention. The solvent, the activator and the reactive oxygen species are mixed. The solution is applied immediately to a toxant by physical associate. If desired, the level of remaining toxant can be determined, and if the toxant is not sufficiently decontaminated (i.e., greater than about 99.4% is removed or neutralized), the steps can be repeated. The activator can comprise more than one activator, and can be liquid or dry, or a combination thereof.

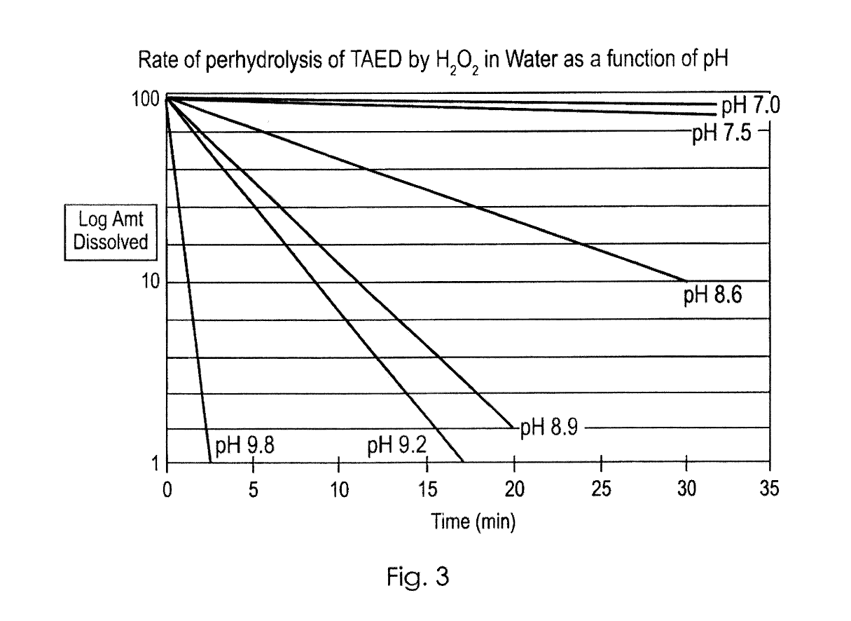

One method of activating the decontaminant formulations of the present invention is to generate the peroxy-oxidants using peroxides as activators which generate peroxycarboxylic acids from their dry or dissolved sources. Such a system can be based on the use of tetraacetyl ethylenediamine (TAED) or tetraacetylmethylenediamine (TAMD) as the peroxyacetic acid source. The activator in such a system can be based upon the use of dry hydrogen peroxide sources, such as urea peroxide, sodium perborate, or sodium percarbonate, which can serve as a buffering agent in an appropriate buffer system to create the quasi-steady state equilibrium of the present invention. Activators provide buffering action for the system. The buffering action of the activators can also be due to carbonate compounds. The function of a buffering agent is generally to drive an acidic or basic solution to a certain pH and then, through the buffering capacity of the solution, prevent a change in the pH. FIG. 3 shows the rate of perhydrolysis of TAED by H₂O₂ in unbuffered water as a function of pH. In the present invention, the organic/aqueous solutions, the reactive oxygen species and their sources, and the activators are part of a carefully balanced buffer solution which establishes a quasi-steady state equilibrium between the generation of peroxy acids and the buffering acid and its conjugate base.

In one aspect of the present invention, the buffer capacity of a decontaminant formulation is established by the concentrations of an acid and its conjugate base to create a buffer solution which will resist a change in pH as the peroxycarboxylic acids are generated from their sources. One skilled in the art would know that the buffering capacity of the decontaminant system should be selected from those buffer solutions which have the midpoint of their titration curves at the optimum of the percarboxylic acid activation curve. One novel discovery that made the present invention possible was the discovery that the isomers of some solvents materially affect the buffering capacity of some buffer systems. The isomers include isomers of butanediol and linear or branched-chain alcohols, including but not limited to, linear or branched alcohols with from 1 to at least 15 carbon atoms. Use of these isomers is discussed further below.

In one preferred form of the present invention, sodium carbonate is used to generate a bicarbonate buffer system in select organic/aqueous solutions comprising certain solvents from Groups II and IV (See Table 2), where the rate at which hydrogen peroxide is released from one or more peroxide sources is used to modulate the perhydrolysis of peroxyacetic acid from TAED, thereby creating the quasi-steady state equilibrium of the decontaminants of the present invention.

Of particular importance to the present invention is that hydrogen bonds account for the unusual properties of water, such as its high boiling point, its large solvency for ionic and polar solutes, and its low vapor pressure. In addition, the asymmetry of the water molecule leads to a dipole moment in the symmetry plane pointed toward the more positive hydrogen atoms, enabling each water molecule to enter into multiple hydrogen bonds at any given moment. The exact number of hydrogen bonds in which a molecule in liquid water participates fluctuates with time and depends on the temperature. From molecular modeling of liquid water at 25° C., it has been estimated that each water molecule participates in an average of 3.59 hydrogen bonds. At 100° C., this number decreases to 3.24 due to the increased molecular motion and decreased density, while at 0° C., the average number of hydrogen bonds increases to 3.69. In order for a compound such as an organic solvent or a chemical agent to dissolve significantly in water, it must disrupt the hydrogen bonds between water molecules. This is formally characterized by Pauling's second rule, which is discussed below.

For the purpose of the present invention, the term “hydrogen bond” can be illustrated by single water molecule (H₂O) in a V-shape, but because the oxygen atom is more electronegative than the hydrogen atoms, the electrons in the molecule tend to gather toward the oxygen end, creating a slightly negative pole with a corresponding slightly positive pole at each hydrogen. This asymmetry of the water molecule leads to a dipole moment in the symmetry plane pointed toward the more positive hydrogen atoms. The measured magnitude of this dipole moment can be calculated:

p=6.2×10⁻³⁰ C·m

This polarity creates a dipole-dipole bond, or hydrogen bond, between water molecules. More generally, a hydrogen bond is a type of “dipole-dipole bond.” The term “dipole-dipole bond” relates to any solvent which has a large dipole moment, such as the strong dipole of a water molecule, which can enter into dipole-dipole bonds with such polar aprotic solvents as nitriles. Acetonitrile, for example, has a very strong dipole moment that can readily enter into dipole-dipole bonds with water molecules. Hence, despite being a Group I polar aprotic solvent, acetonitrile is soluble in water in all proportions.

The invention relates to certain subsets of the possible organic/aqueous solutions comprising at least one polar amphipathic solvent which can form dipole-dipole bonds, including hydrogen bonds with water. In chemistry, a polar-protic solvent is a solvent that has a hydrogen atom bound to an oxygen as in a hydroxyl group or a nitrogen as in an amine group, or, more generally, any molecular solvent which can donate an H⁺ or proton. Common characteristics of polar, protic solvents include:

solvents display hydrogen bonding

solvents have an acidic hydrogen (although they may be very weak acids)

solvents are able to stabilize and dissolve ions:
cations by unshared free electron pairs
anions by hydrogen bonding

Examples of polar, protic solvents are water, methanol, ethanol, formic acid, butanediol and percarboxylic acids. A polar aprotic solvent shares ion-dissolving power with protic solvents, but lacks an acidic hydrogen. These solvents generally have high dielectric constants and high polarity. Examples of polar, aprotic solvents are acetonitrile, dimethylformamide, methylene chloride, dimethyl sulfoxide, and dioxane.

Polar-protic solvents are favorable for S_{N}1 nucleophilic reactions, while polar aprotic solvents are favorable for S_{N}2 nucleophilic reactions. In an S_{N}2 reaction, the addition of a nucleophile and the elimination of a leaving group take place simultaneously. S_{N}2 reactions can occur when the central carbon atom is easily accessible to the nucleophile. In contrast, an S_{N}1 reaction involves two steps. S_{N}1 reactions tend to be important when the central carbon atom of the substrate is surrounded by bulky groups, because such groups interfere sterically with the S_{N}2 reaction and a highly substituted carbon forms a stable carbocation. Apart from solvent effects, polar aprotic solvents may also be essential for reactions which use strong bases, such as reactions involving Grignard reagents or n-butyllithium. If a protic solvent were to be used, the reagent would be consumed by a side reaction with the solvent.

The organic/aqueous solutions of the present invention comprise one or more organic solvents selected from four (4) separate groups consisting of:
Polar, aprotic solvents that do not contain oxygen but have large dipole moments, such as nitriles, haloalkanes, and amides;
polar, monoprotic solvents that contain one (1) —OH moiety, and have large dipole moments, such as the linear or branched-chain alcohols;
polar, aprotic solvents that contain oxygen but to not have an acidic proton, such as ketones, aldehydes, ethers, furans, and dioxins; and
polar, polyprotic solvents (collectively, “polyols”) that contain two or more —OH moieties, and have large dipole moments, such as the diols, triols and higher order polyols.

Hybrid solvents that have the properties of two or more groups are included within the scope of the present invention. Exemplary hybrid solvents include 2-butoxyethanol, cyanocarboxylic acids, butanoic acid, and ethyl acetate. The organic/aqueous solutions of the present invention are further restricted to solvent mixtures wherein the volume fraction of water in the solution ranges from about 25% to about 75%, and the final pH of the solution is less than or equal to about 8.5. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

Exemplary solutions include the alkanediol/water solutions, comprising one of the isomers of propanediol, butanediol, or pentanediol, and solutions made from linear monoprotic alkanols, such as ethanol or butanol or a hybrid of two solvent types such as 2-butoxyethanol. The linear or branched-chain monoprotic alkanols can have from 1 to at least 15 carbons. Additional properties of the organic/aqueous solutions of the present invention should preferably include:
the ability to dissolve, as a single isotropic solutions, full threat loads of CBW toxants, where a chemical threat load is 10 mg/m² or 10 mg/m³ and a biological threat load is 10⁸ particles (cfu or pfu) per mL;
the capacity to dissolve sufficient amounts of polar perhydrolysis agents to enable stoichiometric perhydrolysis of the dissolved toxant threat load;
a low fluid viscosity over the temperature range −25° F.≦T≦125° F.;
a low vapor pressure and a high Flash Point at temperatures >140° F.; and
the ability to be sprayed onto a contaminated surface without having the composition of the decontaminant solution change.

Decontamination solutions of the present invention may be effectively deployed in neutralizing toxants over a temperature range of between about −25° F. and about 140° F. Preferably, the decontamination solutions may be deployed and dispersed as aerosolized sprays over the range of temperatures of between about −25° F. and about 140° F. Other embodiments within the scope of the invention include decontaminants with improved hydrolytic activity at about −25° F., but which do not rapidly evaporate at temperatures as high as about 125° F. Rapid evaporation would limit the effectiveness of the decontamination solution and thus should be avoided, if possible. The solutions may also be applied directly to a surface by pouring or otherwise applying the solution to the surface. The working temperature range for decontamination can be adjusted by selection of the type and relative amounts of polar amphipathic solvents in organic/aqueous solutions according to the total amount of peroxy-oxidant can vary according to the embodiments of the invention. One skilled in the art having the present specification as their guide would know that the solutions made using polyprotic acids, such as the Group IV solvents, will have an entirely different buffering capacity as compared to solutions which comprise monoprotic solvents from Group II. Similarly, it will be known to one skilled in the art having the present specification as their guide that all equilibrium constants vary with temperature according to the van't Hoff equation, and that some solvents will be more likely to promote ionization of a dissolved acidic molecule if the organic/aqueous solution comprises:
a protic organic solvent, capable of forming hydrogen bonds;
a solvent with a high donor number, making it a strong Lewis base; and/or
a solvent with a high dielectric constant, making it a good solvent for ionic species.

Dimtheylsulfoxide (DMSO) has a lower dielectric constant than water, is less polar, dissolves non-polar, hydrophobic substances more readily, and has a measurable pKₐ range of about 1 to 30. Acetonitrile is less basic than DMSO. Also, acids are generally weaker and bases are generally stronger in this solvent. Some pKₐ values at 25° C. for acetonitrile and dimethyl sulfoxide (DMSO) are shown in the Table 5, where values for water are included for comparison.

TABLE 5
pKₐ values of acids in Group I (acetonitrile),
Group III (DMSO) and Group II solvents

| | | | Water |
|---|---|---|---|
| | Acetonitrile | DMSO | (for comparison) |
| HA A⁻ + H⁺ | | | |
| p-Toluenesulfonic | 8.5 | 0.9 | strong |
| acid | | | |
| 2,4-Dinitrophenol | 16.66 | 5.1 | 3.9 |
| Benzoic acid | 21.51 | 11.1 | 4.2 |
| Acetic acid | 23.51 | 12.6 | 4.756 |
| Phenol | 29.14 | 18.0 | 9.99 |
| BH⁺ B + H⁺ | | | |
| Pyrrolidine | 19.56 | 10.8 | 11.4 |
| Triethylamine | 18.82 | 9.0 | 10.72 |
| Proton sponge | 18.62 | 7.5 | 12.1 |
| Pyridine | 12.53 | 3.4 | 5.2 |
| Aniline | 10.62 | 3.6 | 9.4 |

There are many factors that affect pKₐ values. For example, Pauling's second rule states that the value of the first pKₐ for acids of the formula XOₘ(OH)ₙ is approximately independent of n and X and is approximately 8 for m=0, 2 for m=1, −3 for m=2 and <−10 for m=3. This correlates with the oxidation state of the central atom, X: the higher the oxidation state the stronger the oxyacid. For example, pKₐ for HClO is 7.2, for HClO₂ is 2.0, for HClO₃ is −1 and HClO₄ is a strong acid. With organic acids like the percarboxylic acids, inductive effects and mesomeric effects affect the pKₐ values. A simple example is provided by the effect of replacing the hydrogen atoms in acetic acid by the more electronegative chlorine atom. The electron-withdrawing effect of the substituent makes ionization easier, so successive pKₐ values decrease in the series 4.7, 2.8, 1.3, and 0.7 when 0, 1, 2 or 3 chlorine atoms are present. The Hammett equation, provides a general expression for the effect of substituents:

log Kₐ=log Kₐ⁰+ρσ

where Kₐ is the dissociation constant of a substituted compound, Kₐ⁰ is the dissociation constant when the substituent is hydrogen, ρ is a property of the unsubstituted compound, and σ has a particular value for each substituent. A plot of log Kₐ against σ is a straight line with intercept log Kₐ⁰ and slope ρ. This is an example of a linear free energy relationship as log Kₐ is proportional to the standard fee energy change. Hammett originally formulated the relationship with data from benzoic acid with different substituents in the ortho- and para-positions: some numerical values are in Hammett equation. This and other studies allowed substituents to be ordered according to their electron-withdrawing or electron-releasing power. These allow a solution to be tailored for the toxants to be decontaminated and their location.

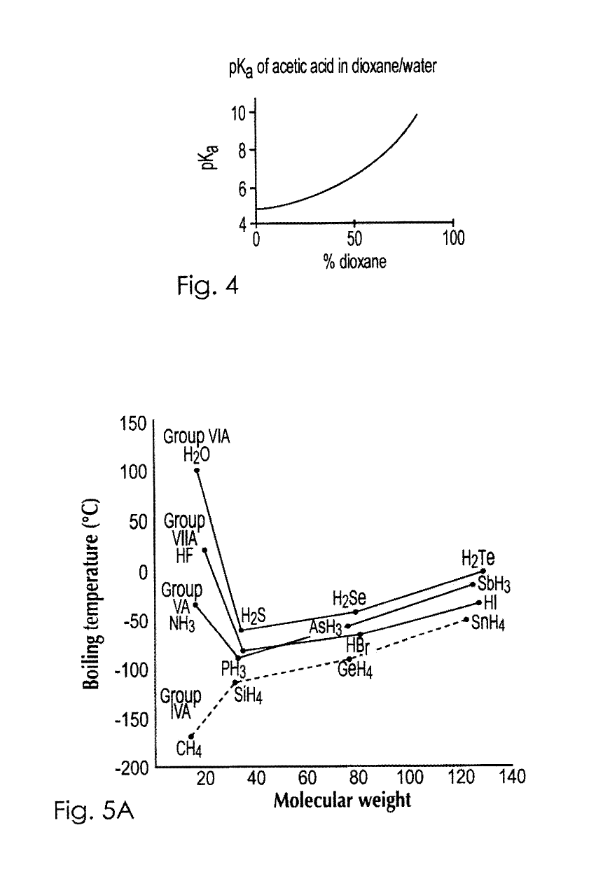

One aspect of the present invention is the use of mixed solvents to create decontaminant formulations which can dissolve threat loads of amphipathic compounds like the nerve or mustard agents and their respective simulants that have limited solubility in water. It is a common practice (in the pharmaceutical industry, for example) to determine pKₐ values in solvent mixture such as water/dioxane or water/octanol, in which the compound is more soluble. In the example shown FIG. 4, the pKₐ value rises steeply with increasing percentage of 1,4-dioxane, a GROUP III solvent with two hydrogen bond acceptors, as the dielectric constant of the mixture is decreasing. A pKₐ value obtained in a mixed solvent cannot be used directly for aqueous solutions, because when the solvent is in its standard state, its activity is defined as one.

For example, the standard state of water:dioxane 9:1 is precisely that solvent mixture with no added solutes. To obtain the pKₐ value for use with aqueous solutions it has to be extrapolated to zero co-solvent concentration from values obtained from various co-solvent mixtures. These factors are frequently forgotten by those having ordinary skill in the art, owing to the omission of the solvent effect from the expression for acid dissociation constants. This is normally used to define pKₐ, but pKₐ values obtained in a given mixed solvent can be compared to each other, giving relative acid strengths. The same is true of pKₐ values obtained in a particular non-aqueous solvent such a DMSO. As of November of 2008, no universal, solvent-independent scale for acid dissociation constants had been developed, since there was no known way to compare the standard states of two different solvents. Given that there is no basis for any type of comparison between solvents, the pKₐ values of the peroxyacids of the present invention and the buffer systems for chemical activation are novel to the present invention.

The total amount of peroxy-oxidant used can vary according to the embodiments of the invention. The total decontaminant to toxant ratio can range from about 100 to about 0.1. The solutions of the invention can be used for hydrolyzing a substrate in an organic/aqueous solution by providing both the toxant and the oxidizers with an isotropic solution in which to react. According to another embodiment of the invention, toxant hydrolyses can be conducted from between about −35° F. and 140° F. In another embodiment of the invention, substrate hydrolysis reactions are conducted between about −25° F. and about 125° F. The total decontaminant to toxant ratio by volume can range from about 100 to about 0.1.

Biopathogens used for biological warfare, such as bacterial cells, bacterial spores, viruses, and other biopathogens have certain structural features that must be considered. The organization and structure of phospholipids and proteins in cell membranes, spore coats and viral capsids can be readily disrupted and hydrolyzed by the organic/aqueous solutions and reactive oxygen species of the invention. These solutions can destroy the integrity of the membranes, spore coats, and viral capsids, exposing the proteins and nucleic acids within the pathogen. The destruction of cellular organelles can lead to neutralization of cell-based biological threats (e.g., bacterial endospores). In this aspect, neutralization includes permanently eliminating the infectivity or toxicity of bacteria, bacterial spores or viruses.

Other embodiments of the invention provide methods of decontaminating toxants using a decontamination solution comprising an organic/aqueous solution containing water-soluble polar amphipathic organic solvents, as described above in Table 2. Suitable polar aprotic solvents include, but are not limited to, those of Groups I through IV. These include nitriles, ketones, dimethyl sulfoxide, and tetrahydrofuran. Suitable polar-protic solvents include, but are not limited to, alcohols and polyols (e.g., diols, triols and certain complex sugars such as fructose). The volume fraction of water in the composition may range from about 25% to about 75%. In one embodiment, the final solution pH has a value less than or equal to about 8.5 but is determined by the buffering capacity of the buffer system. A solution, such as the solutions of the invention, contains both acid and its salt, and has a titration curve, which has a mid-point at which a certain pH can be maintained. In order to maintain the pH of a solution, a buffer that has a mid-point at the desired pH would be selected. Additional embodiments of the invention provide methods of decontaminating toxants, which include phosphoric acid esters, sulfur mustards, bacteria, bacterial spores and viruses. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

Another embodiment of the invention relates to methods of aerosolizing decontamination solutions by dispersing the organic/aqueous decontaminant formulations through a nozzle as a microemulsion. In certain preferred embodiments, decontamination solutions may be deployed over a temperature range of between about −35° F. to about 140° F. In a preferred embodiment, the decontamination solutions of the invention may be deployed at a temperature of between about −25° F. to about 125° F. The working temperature range can be adjusted by varying the type and relative amounts of polar amphipathic solvents in the aqueous organic solutions. The total amount of peroxy-oxidant can vary according to embodiments of the invention. The total decontaminant (and hence oxidant) to toxant ratio can range from about 100 to about 0.1 by volume. Because of the potential for corrosion, the nozzle, if employed, should preferably be made of stainless steal or a thermoplastic material.

One embodiment of the invention encompasses isotropic organic/aqueous solutions comprising at least two polar amphipathic organic solvents, which can be water-soluble and selected from the four distinct solvent groups as set forth in Table 2. Examples of these solutions include but are not limited to those given in Tables 6A-6D below. According to certain aspects of the invention, methods of decontaminating toxants may use a decontamination solution comprising an organic/aqueous solution containing at least two polar amphipathic organic solvents.

Another embodiment of the invention encompasses organic/aqueous solutions comprising at least two polar amphipathic organic solvents, which may be water-soluble and which may further comprise at least one fully dissolved reactive oxygen species, or at least one oxidizers and an activator. Suitable oxidizers, or activators include, but are not limited to, hydroxyl radicals, hydroperoxide anions, superoxides, hydronium ions, peroxyacetic anions, and singlet oxygen.

The terms “amphiphile” and “amphipath” describe chemical compounds possessing both hydrophilic and hydrophobic properties. The hydrophobic group is typically a large hydrocarbon moiety, such as a long chain of the form CH₃(CH₂)ₙ, with n>4. The hydrophilic group can fall into one of the several categories. First, the hydrophilic group can be a charged group, which can be anionic or cationic. Anionic groups are positively charged, and can be carboxylates, sulfates, sulfonates and phosphates. Phosphate esters can be part of an amphipathic compounds and contain a charged functionality as in phospholipid compounds and nerve agents. Cationic groups have a negative charge, and are exemplified by amines. Alternatively, hydrophilic groups can be polar, uncharged groups. Examples of polar, uncharged groups include alcohols with large R groups, such as diacyl glycerol (DAG) and oligoethyleneglycols with long alkyl chains.

Often, amphiphilic species have several hydrophobic parts, several hydrophilic parts, and/or several of both. Proteins and some block co-polymers are examples of such compounds. The hydrophobic regions of these compounds are usually of hydrocarbon nature. The hydrophilic regions are generally represented by either ionic or uncharged polar functional groups. As a result of having both hydrophobic and hydrophilic structural regions, some amphiphilic compounds may dissolve in water, and to some extent in non-polar organic solvents. The organic/aqueous solutions of the present invention, when placed in an immiscible biphasic system consisting of aqueous and hydrophobic solvent, will partition the two phases. The balance between hydrophobic and hydrophilic natures defines the extent of partitioning.

In certain embodiments of the present invention, methods are provided for decontaminating toxants which include organophosphate esters, sulfur and nitrogen mustards, bacteria, bacterial spores, and viruses. Examples of the four different Groups of polar organic solvents of the present invention are listed in Tables 6A-6D below. Tables 6A-6D include examples and properties of some solvents used in the organic/aqueous solutions and the decontaminants of the present invention as well as some unsuitable solvents. The melting point (“M.Pt.”) and the boiling point (“B.Pt.”) are the temperatures at which an undiluted compound undergoes its solid-to-liquid and its liquid-to-vapor phase transitions, respectively. For the purposes of this invention, the principal organic component is considered the “solvent,” whereas the water and other components are all considered the “solutes.” The boiling point and the freezing point of an organic/aqueous solution are two colligative properties that are impacted by the deviation of the solution from ideality (i.e., properties that depend on the number of particles, not the mass of the particles, which include, but are not limited to, lowering of vapor pressure, elevation of boiling point, depression of freezing point, and osmotic pressure). Compounds which have weak intermolecular forces in solution tend to have low boiling points, whereas compounds which have strong intermolecular forces in solution tend to have high boiling points.

The term “polar solvent” refers to solvents with large dipole moments and high dielectric constants; those with low dipole moments and small dielectric constants are classified as non-polar (“apolar”). On an operational basis, solvents that are miscible with water are polar, while those that are not are non-polar, as well as solvents lacking the ability to form dipole-dipole bonds, of which hydrogen bonds are a subset. The term “polar-protic solvent” refers to a solvent able to donate a hydrogen bond between its oxygen and another molecule as between a hydroxyl group or a nitrogen, such as found an amine group, and the oxygen in a Group III solvent. Examples of polar-protic solvents include water, C₂-C₁₂ alkanols (e.g., ethanol), diols, and polyols, acetic acid, formic acid, hydrogen fluoride, and ammonia. More generally, any molecular solvent which contains dissociable H⁺, such as hydrogen fluoride, can be considered to be a “protic” solvent. The molecules of such solvents can donate an H⁺ (proton). Polar-protic solvents are favorable for S_{N}1 nucleophilic reactions. Examples of compounds that are hydrogen acceptors but not donors are dimethyl sulfoxide, dimethylformamide, and dioxane. Examples of hydrogen bond donor compounds include 2-butoxyethanol and ethyl acetate, which have oxygens that are both hydrogen bond acceptors, but only one of which is a donor.

Conversely, “aprotic” means solvents that cannot donate a hydrogen atom. One important characteristic of a protic solvent is that it displays hydrogen bonding, both as an acceptor and a donor. This term includes any solvent that has a similar ion-dissolving power as protic solvents, but lacks an acidic hydrogen. These solvents generally have high dielectric constants and high polarity, and can either act as a hydrogen bond acceptor or enter into strong dipole-dipole bonds that need not involve a hydrogen atom bound to an oxygen. Polar aprotic solvents are favorable for S_{N}2 nucleophilic reactions.

The term “dielectric constant,” as used here, refers to the relative static permittivity, or static relative permittivity, of a material under given conditions. The dielectric constant is a measure of the extent to which a material concentrates electrostatic lines of flux. It is the ratio of the amount of stored electrical energy when a potential is applied, relative to the permittivity of a vacuum. The relative static permittivity is the same as the relative permittivity evaluated for a frequency of zero. The strength of the hydrogen bonds formed by solvent isomers having the same chemical composition is significantly impacted by the number and positions of the hydrogen bond donor and acceptor atoms. For example, the four isomers of butanediol shown in Table 7, all have the same chemical composition, C₄H₁₀O₂, but differ significantly in their molecular structures, boiling points, melting points, and flash points. It is noted that two of the butanediol isomers, 1,2- and 2,3-butanediol, have significantly lower flash points than the 1,3- and 1,4-isomers indicating weaker intermolecular hydrogen bonding by the former.

TABLE 6A
Examples of Group I Polar Aprotic Organic Solvents and their Physical Properties

| | | | | | | | Density |
|---|---|---|---|---|---|---|---|
| | Chemical | B. Pt. | | Dielectric | Flash | | g/ml @ |
| Solvent | Formula | M. Pt. | | Constant | Point | | 20° C. |
| Acetonitrile | C₂H₃N | 82° | C. | 37.5 | 2° | C. | 0.786 |
| | | −45° | C. | | | | |
| Propionitrile | C₃H₅N | 97.2° | C. | 29.7 | 6° | C. | 0.7912 |
| | | −91.8° | C. | | | | |
| Butyronitrile | C₄H₇N | 117.5° | C. | 20.7 | 16° | C. | 0.795 |
| | | −112° | C. | | | | |
| Benzonitrile | C₆H₅CN | 191° | C. | 26.0 | 75° | C. | 1.0 |
| | | −13° | C. | | | | |
| Hexanedinitrile | C₆H₈N₂ | 295° | C. | 13 | 113° | C. | 0.97 |
| (adiponitrile) | | 1° | C. | | | | |
| | C₅H₆N₂ | 287° | C. | 37 | 112° | C. | 0.995 |
| Glutaronitrile | | −29° | C. | | | | |
| 4-Methyl | C₆H₈N₂ | 274° | C. | 15.5 | 126° | C. | 0.95 |
| Pentane nitrile | | −45° | C. | | | | |
| 2-Thiophene | C₆H₅SN | 234° | C. | 37.5 | >110° | C. | 0.95 |
| Acetonitrile | | −5° | C. | | | | |
| n-ValeroNitrile | CH₃(CH₂)₃CN | 141° | C. | 17.7 | 40° | C. | 0.795 |
| | | −10° | C. | | | | |
| MandeloNitrile | C₆H₅CH(OH)CN | 170° | C. | 17 | 113° | C. | 1.117 |
| | | 28-30° | C. | | | | |

| | | | | | | |
|---|---|---|---|---|---|---|
| Dichloromethane | CH₂CL₂ | 40° | C. | 9.1 | none | 1.326 |

| | |
|---|---|
| −97° | C. |

| | | | | | | |
|---|---|---|---|---|---|---|
| Carbon | CCl₄ | 76° | C. | 9.08 | 2.23 | 1.594 |
| tetrachloride | | | | | | |

TABLE 6B.1
Examples of Group II Polar Monoprotic Organic
Solvents and Their Physical Properties

| | | | | | | Density | |
|---|---|---|---|---|---|---|---|
| | Chemical | B. Pt | | Dielectric | Flash | @20 C. | |
| Solvent | Formula | M. Pt. | | constant | Point | g/ml | |
| Methanol | CH₃OH | 64.7° | C. | 33 | 11° C. | 0.7918 | g/ml |
| | | −97° | C. | | | | |
| Ethanol | C₂H₅OH | 78.4° | C. | 30 | 13° C. | 0.789 | g/ml |
| | | −114.3° | C. | | | | |
| n-Propanol | C₃H₇OH | 97.1° | C. | 20 | 15° C. | 0.8034 | g/ml |
| | | −126.5° | C. | | | | |
| Isopropanol | C₃H₇OH | 82.3° | C. | 20.18 | 12° C. | 0.786 | g/ml |
| | | −89° | C. | | | | |
| n-Butanol | C₄H₉OH | 117.2° | C. | 18 | 29° C. | 0.8098 | g/ml |
| | | −89.5° | C. | | | | |
| n-Pentanol | C₅H₁₁OH | 138° | C. | 16 | 33° C. | 0.8 | g/ml |
| | | −77.6° | C. | | | | |
| n-Hexanol | C₆H₁₃OH | 158° | C. | 12 | 63° C. | 0.8136 | g/ml |
| | | −46.7° | C. | | | | |
| n-Octanol | C₈H₁₇OH | 195° | C. | 5-10 | 63° C. | 0.824 | g/ml |
| | | −16.° | C. | | | | |
| Formic Acid | CH₂O₂ | 101° | C. | 58 | 69° C. | 1.22 | g/ml |
| | | 8.4° | C. | | | | |
| Acetic Acid (1) | C₂H₄O₂ | 118.1° | C. | 6.2 | 43° C. | 1.049 | g/ml |
| | | 16.5° | C. | | | | |
| Butanoic Acid | C₄H₈O₂ | 163.5° | C. | 3.0 | 72° C. | 0.96 | g/ml |
| | | −7.9° | C. | | | | |
| Hexanoic acid | C₆H₁₂O₂ | 202° | C. | NA | 102° C. | 0.92 | g/ml |
| | | −3° | C. | | | | |

TABLE 6 B.2
Water Solubilities of Group II Linear Monoprotic Alcohols

| | | Water Solubility: | |
|---|---|---|---|
| Solvent | Chemical Formula | g/100 grams H₂O | |
| Methanol | CH₃OH | infinitely soluble | |
| Ethanol | CH₃—CH₂—OH | infinitely soluble | |
| Propanol | CH₃—CH₂—CH₂—OH | infinitely soluble | |
| Butanol | CH₃—CH₂—CH₂—CH₂—OH | 8.88 | grams/100 |
| Pentanol | CH₃—CH₂—CH₂—CH₂—CH₂—OH | 2.73 | grams/100 |
| Hexanol | CH₃—CH₂—CH₂—CH₂—CH₂—CH₂—OH | 0.602 | grams/100 |
| Heptanol | CH₃—CH₂—CH₂—CH₂—CH₂—CH₂—OH | 0.174 | grams/100 |

| | | |
|---|---|---|
| Octanol | CH₃—CH₂—CH₂—CH₂—CH₂—CH₂—CH₂OH | insoluble |

TABLE 6C.1
Examples of Group III Polar Aprotic Solvents Containing Oxygen and Physical Properties

| | | B. Pt | | Dielectric | Flash | | Dens.@20 C. |
|---|---|---|---|---|---|---|---|
| Solvent | Chemical Formula | M. Pt. | | constant | Point | | g/ml |
| 1,4-dioxane | /—CH₂—CH₂—O—CH₂—CH₂—O—\ | 101° | C. | 2.2 | 12° | C. | 1.033 |
| | | 11.8° | C. | | | | |
| 1,3-dioxane | C₄H₈O₂ | 102° | C. | NA | 2° | C. | 1.03 |
| | | −42° | C. | | | | |
| Tetrahydrofuran | /—CH₂—CH₂—O—CH₂—CH₂—\ | 66° | C. | 7.58 | −14° | C. | 0.886 |
| | | −108° | C. | | | | |
| Acetone | CH₃—C(═O)—CH₃ | 56° | C. | 20.7 | −17° | C. | 0.786 |
| | | −94.9° | C. | | | | |
| Acetophenone | C₆H₅C(O)CH₃ | 202° | C. | 17.3 | 82° | C. | 1.028 |
| | | 20° | C. | | | | |
| Benzophenone | C₁₃H₁₀O | 305° | C. | NA | 143° | C. | 1.11 |
| | | 47.9° | C. | | | | |
| Formamide | CH₃NO | 210° | C. | 84.0 | 154° | C. | 1.133 |
| | | 2° | C. | | | | |
| Dimethylformamide | H—C(═O)N(CH₃)₂ | 153° | C. | 38 | 57.8° | C. | 0.944 |
| | | −61° | C. | | | | |
| Dimethylsulfoxide | C₂H₆OS | 189° | C. | 48.0 | 89° | C. | 1.1004 |
| | | 18.5° | C. | | | | |
| 1,3-Dimethyl- | C₅H₁₀N₂O | 225° | C. | 37.60 | 120° | C. | 1.05 |
| 2-Imidazolidinone | | 8.2° | C. | | | | |
| Methyl Ethyl Ketone | C₄H_{8O} | 79.6° | C. | 18.4 | −7° | C. | 0.805 |
| | | −86° | C. | | | | |
| Diethyl ether | CH₃CH₂—O—CH₂—CH₃ | 35° | C. | 4.3 | −45° | C. | 0.713 |
| | | −116° | C. | | | | |

TABLE 6 C.2
Examples of Group II/Group III Polar Hybrid Solvents and Their Physical Properties

| | | B. P | | Dielectric | Flash | | Density |
|---|---|---|---|---|---|---|---|
| Solvent | Chemical Formula | M. Pt. | | Constant | Point | | g/ml |
| 3-Methoxy- | CH₃CH(OCH₃)CH₂CH₂OH | 161° | C. | NA | 46° | C. | 0.922 |
| 1-Butanol | | −85° | C. | | | | |
| 2-Butoxy- | C₆H₁₄O₂ | 171° | C. | 9.3 | 67° | C. | 0.90 |
| Ethanol | | −70° | C. | | | | |
| 2-Butoxy- | C₄H₉O(CH₂)₂OCOCH₃ | 192° | C. | NA | 71.1° | C. | 0.94 |
| Ethanol Acetate | | −63.3° | C. | | | | |
| Ethyl Acetate | CH₃—C(═O)—O—CH₂—CH₃ | 77° | C. | 6.02 | −4° | C. | 0.894 |
| | | −83.6° | C. | | | | |
| Diacetone | CH₃C(═O)CH₂C(OH)(CH₃)₂ | 166° | C. | NA | 58.8° | C. | 0.938 |
| Alcohol | | −47° | C. | | | | |

TABLE 6 D
Examples of Group IV Polar Polyprotic Solvents and Their Physical Properties

| | | | Dielectric | Flash | | Density |
|---|---|---|---|---|---|---|
| | B. P | | Constant | Point | | g/ml |
| Solvent | M. Pt. | | @ 20° C. | closed cup | | @ 20° C. |
| Ethylene Glycol | 37-41.2° | C. | 11.9 | 110° | C. | 1.113 |
| | −13° | C. | | | | |
| 1,2-Propanediol (Propylene Glycol) | 188.2° | C. | 32.0 | 107° | C. | 1.036 |
| | −59° | C. | | | | |
| 1,3-Propanediol | 210° | C. | 29 | 79° | C. | 1.053 |
| | −28° | C. | | | | |
| 2,2-Dimethyl 1,3-Propanediol | NA | | 31 | 103° | C. | NA |
| | 126° | C. | | | | |
| 2,3-Butanediol | 184° | C. | 4 | 85° | C. | 0.995 |
| | 25° | C. | | | | |
| 1,3-Butanediol | 207.5° | C. | 28.8 | 108.9° | C. | 1.005 |
| | <−50° | C. | | | | |
| 1,4-Butane diol | 230° | C. | 31.9 | 134° | C. | 1.017 |
| | 16° | C. | | | | |
| 1,3-Pentanediol | 232° | C. | 16 | 113° | C. | 0.98 |
| | 52-56° | C. | | | | |
| 1,5-Pentanediol | 242° | C. | 26.2 | 136° | C. | 0.99 |
| | NA | | | | | |
| 1,2-Pentanediol | 206° | C. | 17 | 105° | C. | 0.971 |
| | NA | | | | | |
| Hexylene Glycol | 197° | C. | 23.4 | 90° | C. | 0.92 |
| | −40° | C. | | | | |
| 1,2,6-Hexanetriol | 178° | C. | 31.5 | 79° | C. | 1.109 |
| | NA | | | | | |
| Ethenol (PVA) | 228° | C. | NA | 79.44° | C. | 1.19-1.31 |
| | 230° | C. | | | | |
| 2-(Hydroxyethoxy) | 245° | C. | 6-10 | 123° | C. | 1.118 |
| ethan-2-ol | −10° | C. | | | | |
| (Diethylene Glycol) | | | | | | |
| 2-(2-Hydroxyethoxy) | 127° | C. | 5-10 | 166° | C. | 1.124 |
| ethan-2-ol | −7° | C. | | | | |
| (Triethylene Glycol) | | | | | | |

In order to solubilize and decontaminate chemical and biological agents, hydrogen bonds in the solution must be broken. At first glance then, it might appear desirable to prepare the decontamination solution using organic solvents having weaker hydrogen bonds. Of the four isomers of butanediol, however, it is one of the novel discoveries of the present invention that the oxygens of 1,3-butanediol have higher pKₐ values compared to the other butanediol isomers, enabling the use of this isomer with a different buffer system, and hence a different type of chemical activation in creating a decontaminant formulation. This discovery has proven important to developing the preferred decontamination solution and formulation.

TABLE 7
Boiling Points and Flash Points of Butanediol Isomers

| Isomer | Chemical Structure | Boiling point | Flash Point |
|---|---|---|---|
| 1,4-butanediol | HO(CH₂)₄OH | 230° C. | 134° C. |
| 1,3-butanediol | CH₃CH(OH)CH₂CH₂OH | 203° C. | 121° C. |
| 1,2-butanediol | CH₃CH₂CH(OH)CH₂OH | 191° C. | 93° C. |
| 2,3-butanediol | CH₃CH(OH)CH(OH)CH₃ | 183° C. | 85° C. |

The present invention is intended for use in commerce, which means it must be safe to transport on commercial trucks, trains, and airplanes. The flash point of a compound is one indication of how easily a compound or a solution may burn, or the minimum temperature at which a liquid gives off vapor within a test vessel in sufficient concentration to form an ignitable mixture with air near the surface of the liquid. Chemicals with higher flash points have lower vapor pressures and are less hazardous than chemicals with lower flash points. The flash point of a chemical or a solution is the lowest temperature at which it will evaporate enough fluid to form a combustible concentration of gas, and thus is an indication of how easily a chemical may burn. The Code of Federal Regulations (49 C.F.R. §173:120) identifies ranges of flash points of materials that are characterized as “flammable,” “combustible,” and “non-combustible” compounds. Non-combustible compounds are typically safer than combustible and flammable compounds. A flammable liquid has a flash point of not more than 60° C. (140° F.); a combustible liquid has a flash point above 60° C. (140° F.) and below 93° C. (200° F.). That said, as an example, decontaminants made using the isomers of butanediol are considered safe for all forms of transport without special handling.

The invention relates to methods of decontaminating toxants using a decontaminant comprising an organic/aqueous solution containing at least one polar solvent, which formulation is distinguished by a flash point in organic aqueous solution >140° F. This allows for the creation of decontaminants with low vapor pressures and high flash points that are safe to ship, store, and use over the temperature range of 125° F. to −25° F.

The impact of hydrogen bonding on the flash point of a decontaminant is important in selecting the aqueous/organic mixtures of the present invention. Table 8 shows that two butanediol isomers and a branched propanediol, all of which have the same molecular composition, C₄H₁₀O₂, and which differ only in the separations of their hydroxyl groups, nonetheless have significantly different flash points. Those differences notwithstanding, in terms of flammability and transportation safety, all three diols are acceptable solvents for a deployable decontaminant; whereas among the monoprotic solvents of Table 6B, there are no high flash point alcohol solvents.

TABLE 8
Structure and Flash Point of Butanediol and Propanediol Compounds

| Isomer | Structure | Flash Point |
|---|---|---|
| 1,4-butanediol | 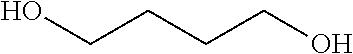 | 134° C. |
| 2,3-butanediol | 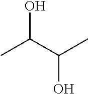 | 85° C. |
| 2-methyl-1,3- Propanediol | 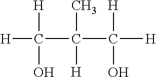 | 127° C. |

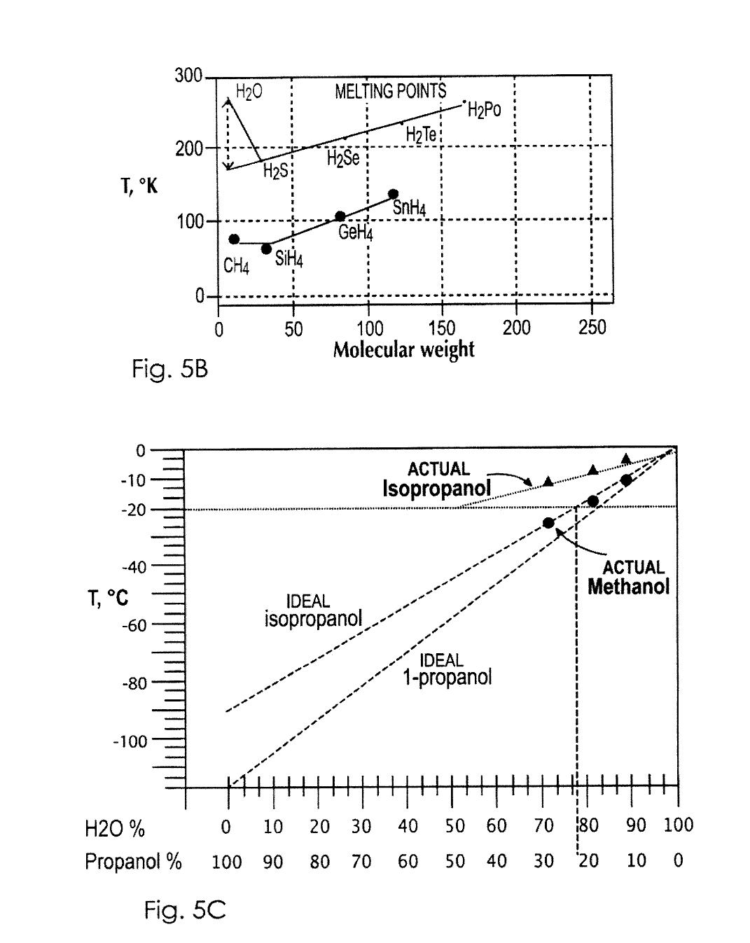

Turning to FIGS. 5A-5C, the boiling point and melting points of water are uniquely elevated relative to other compounds that can form hydrogen bonds. FIG. 5A depicts the boiling temperature of various compound, as compared to the molecular weight. In the upper curve of FIG. 5B, the melting points of molecules that are structurally related to water, which otherwise differ primarily in their molecular masses, shows that the melting points of the series can be predicted from the molecular mass. The sole exception is water, which has a melting point approximately 100° C. higher than would have been predicted from molecular mass alone. The lower curve compares the corresponding structural analogs in which the central atom is drawn from compounds that do not significantly form hydrogen bonds. This shows that the ability to form hydrogen bonds contributes significantly to the stability of what is often referred to as the liquid crystalline structure of water. With reference to FIG. 5C, the actual and ideal freezing points of various aqueous/alkanol solutions are compared and illustrate: (i) the lowering of the freezing point with increasing alkanol fraction; (ii) that hydrogen bonding creates non-ideal solution behavior compared to the expected ideal behavior; and (iii) that the position of the hydrogen bond donor/acceptor on the alkyl chain of the alkanol significantly alters the colligative properties of the solutions.

The concept of an ideal solution is fundamental to chemical thermodynamics and its applications, including the use of colligative properties. In ideal solutions, the role of intermolecular interactions such as hydrogen bonding can be ignored because they are small or because components in the solutions have the same interaction with each other that they have with themselves. Similar solvents will form ideal solutions and their properties are adequately described by Raoult's Law, which states that “[t]he vapor pressure of an ideal solution is dependent on the vapor pressure of each chemical component and the mole fraction of the component present in the solution.” However, in many cases, intermolecular interactions can cause deviations from Raoult's Law.

Decontamination solutions can be optimized for low temperature deployment using the following assumptions: (a) ΔT=k_{f}m from Raoult's Law, (b) for an aqueous solution the freezing point depression=0° C.−ΔT, and (c) i=total moles of ions after solution/moles of solute before solution. However, in contrast to ideal solutions, where volumes are strictly additive and mixing is always complete, the properties of a non-ideal solution are not generally the simple sum of the properties of the component pure liquids. As such, the solubility of a component is not guaranteed over the entire composition range. For example, if the molecular interactions between two components of a solution are more attractive than those between the individual compounds themselves, the vapor pressure above a solution will be smaller than would be calculated using Raoult's law. This in turn would mean a higher flash point and boiling point. Conversely, if the unlike-molecule interactions are more repulsive, then the vapor pressure would be greater than for the corresponding ideal solution. In addition, the flash point and boiling point would be lower.

The organic/aqueous formulations of the present invention can be non-ideal solutions, in which the strong hydrogen bonds of the water fraction are replaced by interactions with the organic solvents. This usually results in changes in the colligative properties, including but not limited to, the boiling and freezing points, vapor pressure, and flash point.

According to certain aspects of the invention, methods of decontaminating toxants use a decontamination solution comprising an aqueous/organic solution containing at least two polar amphipathic organic solvents (at least one aprotic and one protic solvent in combination or at least two protic solvents in combination). The solvents are distinguished by having either: (i) strong dipole moments but contain no oxygen atom; (ii) by their capacities to act as hydrogen bond acceptors; or (iii) by the extent to which they can act as both hydrogen bond donors and acceptors. Suitable polar amphipathic solvents are included in the four groups discussed above and shown in Table 2, with examples given in Tables 6A-6D.

According to certain aspects of the invention, methods of decontaminating toxants use a decontamination solution comprising an organic/aqueous solution containing at least two water-soluble polar amphipathic organic solvents, in which the melting point of the solution is lowered compared to the melting point of water. It is one aspect of the solvent selection process of the present invention that the organic solvents selected for use in decontaminants should have a melting point in the neat, or undiluted, solution that is lower that the melting point of water, and in fact should have a melting point lower than −25° F. Similarly, the boiling point of the organic solvent in the neat solvent should be sufficiently elevated to allow for decontaminant solutions that are in liquid form over the range of about 125° F. to about −25° F. See FIGS. 5A-5C.

Decontaminant solutions can be prepared comprising an organic/aqueous solution containing at least one polar amphipathic organic solvent and water in which the melting point of the solution is lowered compared to the melting point of water. The boiling point remains sufficiently elevated enabling the creation of decontaminant solutions that remain fluid and do not freeze or boil over the temperature range of about 125° F. to about −25° F. Methods of using such decontamination solutions are encompassed by the invention.

According to certain aspects of the invention, methods of decontaminating toxants use a decontamination solution comprising an organic/aqueous solution containing at least two polar amphipathic organic solvents that are dissolved in water as a homogeneous, single-phase solution that remains liquid over the temperature range of about −25° F. to about 125° F.

According to certain aspects of the invention, methods of decontaminating toxants use a decontamination solution comprising an organic/aqueous solution containing at least two water-soluble polar amphipathic organic solvents that are dissolved in water as a homogeneous, single-phase isotropic solution, which is capable of dissolving at least one threat load of a toxant in a homogeneous, isotropic solution, and which remains liquid over the range of about −25° F. to about 125° F.

According to certain aspects of the invention, methods of decontaminating toxants use a decontamination solution comprising an organic/aqueous solution containing at least two water-soluble polar amphipathic organic solvents that are dissolved in water as a homogeneous, isotropic solution, which solution: (i) is capable of dissolving at least one threat load of a toxant in a homogeneous isotropic solution; (ii) is also capable of dissolving reactive oxygen species or their dry sources in sufficient quantities and concentrations to rapidly hydrolyze or otherwise neutralize threat loads of toxants in small volume ratios of decontaminant solution to toxant; and (iii) remains liquid over the temperature range of about −25° F. to 125° F.

According to certain aspects of the invention, methods of decontaminating toxants use a decontamination solution comprising an organic/aqueous solution containing at least two water-soluble polar amphipathic organic solvents (at least one aprotic and one protic solvent in combination or at least two protic solvents in combination) and at least one polyol block co-polymer that are dissolved as a homogeneous, isotropic solution, which solution: (i) is capable of dissolving at least one threat load of a toxant in a homogeneous isotropic solution; (ii) is also capable of dissolving reactive oxygen species or their dry sources in sufficient concentrations to rapidly hydrolyze or otherwise neutralize full threat loads of toxants in small volume ratios of decontaminant solution to toxant; (iii) can be sprayed in high volumes without change of composition; and (iv) can be applied by spraying onto contaminated surfaces where it dissolves toxants to achieve the hydrolysis of dissolved toxants to by-products. This spraying can be as an aerosol, where it persists, and then dissolves aerosolized toxants to achieve the hydrolysis of dissolved toxants to by-products.

The invention relates to solutions in which the solute is dissolved. When aerosolized, the solution forms liquid-in-liquid particles, which are not micellar in nature. The solutions of the invention are completely soluble, isotropic solutions that only form liquid-in-liquid microemulsions that are particulate, when sheared in a nozzle.

In the above aspects of the invention, these decontaminant formulations can be aerosolized to produce a liquid-in-liquid microemulsion (e.g., a microcolloidal system, which has decontaminant and microemulsion components) instead of a homogeneous, single-phase solution. Through the use of appropriate block co-polymers, the liquid-in-liquid microemulsion can also be a non-Newtonian fluid possessing desirable properties as both an aerosol and a surface decontaminant.

Organophosphates can be hydrolyzed when they are dissolved in an aqueous/organic solution comprising polar-protic and/or polar aprotic amphipathic organic solvents, if sufficient molar equivalents of the appropriate activated oxidizer(s) are also dissolved in the formulation. Such solvents are identified in Table 2, including but are not limited to: alkanols, polyols, polar-protic solvents, and polar aprotic amphipathic solvents (e.g., alkanols and nitriles). The addition of certain surface active agents in combination with certain co-polymers to organic/aqueous solutions can be used to dissolve and hydrolyze toxants by converting the solutions to non-Newtonian fluids. The addition of the surface active agents, in particular block co-polymers make it easier to disperse the solutions by spraying. At the same time, the surface active agents improve the ability to form aerosol fogs, as well as to adhere as thin films on surfaces, which increases both the rate and extent of organophosphate and blister agent hydrolysis.

The term “surface active agents” includes the more common term “surfactants” and refers to one or more wetting agents that lower the surface tension of a liquid, allowing easier spreading, and lowering the interfacial tension between two liquids. Surfactants are usually organic compounds that are amphiphilic, meaning they contain both hydrophobic groups (their “tails”) and hydrophilic groups (their “heads”). Block co-polymers have alternating “blocks” that are hydrophilic or hydrophobic and have a more complex surface behavior in which the hydrophobic blocks of the polymer can be solubilized in polar solvents. Whereas the apolar blocks are positioned at an interface, such as an air water interface. Therefore, both conventional surfactants and the block co-polymers can be soluble in both organic solvents and water. Both types of compounds reduce the surface tension of water by adsorbing at the liquid-gas interface. Both types of compounds also reduce the interfacial tension between oil and water by adsorbing at the liquid-liquid interface. Many surfactants and many block co-polymers can also form aggregates in a bulk solution. Examples of such aggregates are vesicles, micelles, and the microemulsions of the present invention, all of which are quite different from one another. The concentration at which surfactants begin to form micelles is known as the critical micelle concentration (“CMC”). Surfactants are also often classified into four primary groups based upon charge: anionic, cationic, non-ionic, and zwitterionic, or dual charge. For the purposes of the present invention, the preferred surfactants are alkanols and the preferred block co-polymers are poly(ethylene oxide) and poly(propylene oxide), e.g., poloxamers or poloxamines. The preferred colloidal form once aerosolized is a non-Newtonian fluid that is also a liquid-in-liquid microemulsion that conforms to the axioms of the general model of decontaminant formulation set forth in this patent application.

The terms “co-polymer,” “block co-polymer,” and “heteropolymer” relate to polymers that are derived from two or more monomeric units, albeit each unit may have a large molecular weight. Block co-polymers are comprised of two or more homopolymer subunits that can be linked by covalent bonds. The union of the homopolymer subunits may require an intermediate non-repeating subunit, which is known as a junction block. Block co-polymers with two or three distinct blocks are called diblock co-polymers and triblock co-polymers, respectively. Block co-polymers can “microphase separate” to form periodic nanostructures, also called “microparticles” or “microsomes”, that are contained within a liquid-in-liquid microemulsion. Because of the microfine structure of the microparticles in such a microemulsion, a microscope or fluorescent label is required to detect and examine the structure of the microparticles.

Block co-polymers of the organic/aqueous mixtures of the invention can be useful for converting organic/aqueous mixtures to “shear-thinning” or “pseudoplastic” non-Newtonian solutions, as described below.

“Microphase separation” refers to a property of solutions when mixing substances such as oil and water, which are normally immiscible. Due to incompatibility between the blocks of an amphipathic block co-polymer, one would expect the compounds to undergo a similar phase separation in the present invention. However, because the blocks are covalently bonded to each other, they cannot demix macroscopically, as do water and oil. In microphase separation, the blocks of the polymers form micrometer-sized structures. Depending on the relative lengths of each block of the polymer, several types of morphologies can be obtained. In the present invention, the blocks can form micron-sized particles. The product of the degree of polymerization, N, and the Flory-Huggins interaction parameter, x, gives an indication of how incompatible the two blocks are and whether or not they will microphase separate.

In general, polymeric mixtures are far less miscible than mixtures of small molecule materials. Miscible materials usually form a solution because of an increase in entropy and free energy associated with increasing the amount of volume available to each component. Conversely, since polymeric molecules are much larger and hence generally have much higher specific volumes than small molecules, the number of molecules involved in a polymeric mixture are far less than the number in a small molecule mixture of equal volume. The energetics of mixing are comparable on a per volume basis for polymeric and small molecule mixtures. This tends to increase the free energy of mixing for polymer solutions and thus make solvation less favorable. Thus, concentrated solutions of polymers are less likely than those of small molecules.

For the organic/aqueous solutions of the present invention, the properties of the polymer can be characterized by the interaction between the solvent and the polymer. In a suitable solvent, the polymer appears swollen and occupies a large volume. Here, intermolecular forces between the solvent and monomer subunits dominate over intramolecular interactions. In a poor solvent, intramolecular forces dominate and the chain can contract. In a theta solvent (also called the Flory condition), the state of the polymer solution where the value of the second virial coefficient becomes zero and the intermolecular polymer-solvent repulsion balances exactly the intramolecular monomer-monomer attraction. Under these conditions, a polymer can behave like an ideal random coil and can form liquid-in-liquid microemulsions.

Some block co-polymers include poloxamers. Poloxamers are nonionic triblock co-polymers composed of a central hydrophobic chain of polyoxypropylene (poly(propylene oxide)) flanked by two hydrophilic chains of polyoxyethylene (poly(ethylene oxide)). Poloxamers include, but are not limited to, the Pluronic® block co-polymers (e.g., Pluronic® F127, Pluronic® F188, Pluronic® 68, and Pluronic® F125). The Pluronic block co-polymers are ethylene oxide and propylene oxide co-polymers. More specifically, the block co-polymer may be an ethylene oxide and propylene oxide co-polymer that terminates in primary hydroxyl groups, one example of which is Pluronic® F127.

Additionally, the block co-polymers can have different ranges of molecular weights. Because of their amphiphilic structure, the polymers have surfactant-like properties that make them useful in industrial and pharmaceutical applications. Among other things, they can be used to increase the water solubility of hydrophobic, oily substances, such as organophosphate esters, pesticides, and nerve agents. These compounds can also increase the miscibility of two substances with different hydrophobicities through the formation of microemulsions. For this reason, these polymers can also be employed in pharmaceutical applications as model systems for slow release drug delivery applications or, as in the present invention, to enhance the solubility of amphipathic toxants and polar reactive oxygen species or their sources in a single isotropic phase.

In one embodiment of the present invention, the triblock co-polymers used in aqueous organic decontamination solutions capable of forming microemulsions are hydrophilic non-ionic triblock co-polymers consisting of a central hydrophobic block of polypropylene glycol flanked by two hydrophilic blocks of polyethylene glycol. The approximate lengths of the two PEG blocks are 100 repeat units while the approximate length of the propylene glycol block is 65 repeat units (see Table 2). The molecular weights of the various triblock co-polymers vary with the number of blocks. Similarly, other such block co-polymers can be made to carry a permanent charge enabling the formation of particles in the microemulsions of the present invention which carry a net positive or negative charge, as desired.

The preferred forms of the co-polymers used in the present invention are the polymers synthesized from the simple alkene ethene, called polyethylenes. These compounds retain the -ene suffix, even though the double bond is removed during the polymerization process.

The attractive forces between polymer chains play a large part in determining a polymer's properties. Because polymer chains are so long, the interchain forces are amplified far beyond the attractions between conventional molecules. Different side groups on the polymer can cause the polymer to tend towards ionic bonding or hydrogen bonding between its own chains. These stronger forces typically result in higher tensile strength and higher melting points. Further, the intermolecular forces in polymers can be affected by dipoles in the monomer units.

The co-polymers of the invention, which can be simple di- or tri-block co-polymers, generally can have alternating hydrophobic and hydrophilic regions. If the block co-polymer has a dipole moment, certain regions of the molecule will gather at the interface between two phases of a biphasic solution. The surface tension will also be reduced, which helps solubilize longer chain alcohols.

Polymers containing amide or carbonyl groups can form hydrogen bonds between adjacent chains because the partially positively charged hydrogen atoms in N—H groups of one chain are strongly attracted to the partially negatively charged oxygen atoms in C═O groups on another. These strong hydrogen bonds can result in the high tensile strength and melting point of polymers containing urethane or urea linkages. Ethene, however, has no permanent dipole. The attractive forces between polyethylene chains arise from weak van der Waals forces. Molecules such as these can be thought of as being surrounded by a cloud of negative electrons. As two polymer chains approach, their electron clouds repel one another, which can cause the electron density on one side of a polymer chain to be lowered, creating a slight positive dipole on that side. This charge can be enough to attract the second polymer chain. Van der Waals forces are weak, however, so polyethylene co-polymers can have a lower melting temperature compared to other polymers. As discussed above, this will also convert the solution to a non-Newtonian solution and lower the viscosity. However, even with a lower viscosity, the solution will stick to a surface better and allow a more efficient decontamination of toxants. In addition, the solution can be sprayed such that the particles are smaller, which also allows better coverage a surface in need of decontamination.

Another aspect of the invention relates to analytical methods based on fluorescent dyes and corresponding hardware to regulate the operation and use of the solution and dispersion system. Fluorescent dyes can be used to detect one or more toxants both before and after treatment with the decontaminating solution, to analyze the mixing, reaction and neutralization of aerosolized toxant in real time, and to analyze the area or volume coverage, extent of toxant neutralization, and elimination of toxant threat during surface decontamination.

Other embodiments of the invention relate to methods of using optoelectronic hardware and software, combined with fluorescent dyes, to regulate the operation and use of the decontamination solution and the dispersal system. As above, such hardware and software can be used to detect one or more toxants both before and after treatment with the decontaminating solution, to analyze the mixing, reaction and neutralization of aerosolized toxant in real time, and to analyze the area or volume coverage, extent of toxant neutralization, and elimination of toxant threat during surface decontamination.

In addition, toxant decontamination can be detected using chromatographic methods including to High Performance Liquid Chromatography (HPLC) and Gas Chromatography (GC) using detectors that employ various detectors, such as absorbance detectors, flame ionization detectors, electron capture detectors, mass spectroscopic detectors, and fluorescence detectors.

The solutions of the invention may be applied directly to a desired surface to be decontaminated or sprayed as an aerosol. In order to prepare organic/aqueous solutions for dispersal using spray nozzles, certain solution parameters should be followed. The flow rate, or capacity of a fluid through a nozzle is affected by a number of factors including pressure, specific gravity, and viscosity of the fluid. The specific gravity, or density, of a liquid represents the ratio of a mass of given volume of liquid to the mass of the same volume of water, as shown by the following equation:

$$LIQUID FLOW = Water flow rate \times \frac{1}{√ specific gravity}$$

Generally, the higher the specific gravity of a liquid the smaller the flow rate of liquid through a nozzle. The viscosity of a liquid is a measure of the resistance to flow. In general, increased pressure is required to atomize more viscous liquids, which results in sprays with a smaller angle, as compared with water alone. Nozzle design governs the extent of this effect, but in general, as viscosity increases, the flow rate of hollow and full cone nozzles is increased, and conversely, the flow of flat sprays are decreased. Surface tension is the condition existing at the free surface of a liquid resembling the properties of an elastic skin under tension. This tension is a result of the intermolecular forces exerting an unbalanced inward pull on the individual surface molecules. Surface tension affects the development of the liquid sheet and hence directly influences minimum operating pressures, droplet size and spray angle. This results in lower surface tension and smaller drops in a mist, which, in turn will have an effect on the application of the decontamination solution to an area containing one or more toxants.

Temperature also influences liquid viscosity, specific gravity, and surface tension, and can have an effect on the performance of spray through a nozzle. Data presented herein are based on aqueous organic liquid applications over the range of temperatures from about −25° F. to as high as about 140° F.

Unidirectional fluid flow, such as through a pipe or nozzle is generally modeled as comprised of layers of fluid flowing past one another. The viscosity of a liquid is a measure of the resistance to flow. In fluid dynamics, Couette flow refers to the laminar flow of a viscous fluid in the space between two parallel plates, one of which is moving relative to the other. The flow is driven by virtue of viscous drag force acting on the fluid and the applied pressure gradient parallel to the plates. This friction becomes apparent when one layer of fluid is made to move in relation to another layer, with the greatest resistance to flow being found at the boundary layer, adjacent to a fixed surface such as the interior wall of a pipe, whereas the lowest resistance and hence the greatest velocity, are at the center as indicated by the arrows in the pipe, as shown in FIG. 20.

The greater the friction between layers in the fluid, the greater the amount of force required to cause this movement, which is called “shear.” Shearing occurs whenever the fluid is physically moved or distributed (e.g., pouring, spreading, spraying, or mixing). Specifically, shear will occur when a fluid is moved through a nozzle in an atomizing spray head, such as an ultrasonic nozzle. Highly viscous fluids require more force to move than less viscous materials.

In a viscosity model, as shown in FIG. 21, two parallel planes of fluid of equal area “A” are separated by a distance “dx” and are moving in the same direction, but at different velocities “V1” and “V2.”

In a Newtonian fluid, the force required to maintain this difference in speed is proportional to the difference in speed through the liquid, or the velocity gradient. The “velocity gradient” is a measure of the change in speed at which the intermediate layers move with respect to each other. It describes the shearing the liquid experiences and is called “shear rate.” This is symbolized as “S” and its unit of measure is called the “reciprocal second” (sec-¹). The term F/A indicates the force per unit area required to produce the shearing action. It is referred to as “shear stress” and is symbolized by “F” with units of measurement in “dynes per square centimeter” (dynes/cm²). Using these simplified terms, viscosity may be defined mathematically by:

$$η = viscosity = \frac{{F}^{′}}{S} = \frac{shear stress}{shear rate}$$

The fundamental unit of viscosity measurement is the “poise.” A material requiring a shear stress of one dyne per square centimetre to produce a shear rate of one reciprocal second has a viscosity of one poise, or 100 centipoise. Viscosity measurements are also occasionally expressed in “Pascal-seconds” (Pas) or “milli-Pascal-seconds” (mPas).

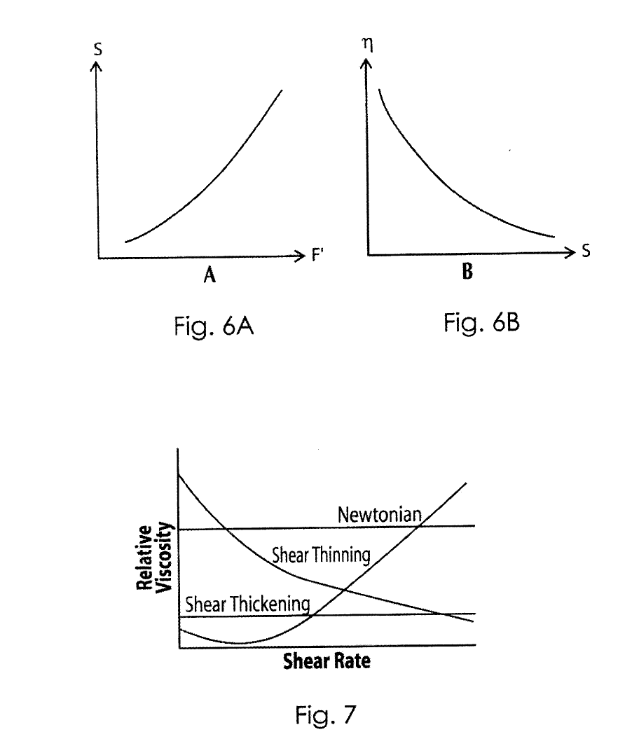

FIGS. 6A-6B relate to a comparison of the effect of shear on different fluids. These figures illustrate the viscoelastic behavior of a type of non-Newtonian fluid designated “pseudoplastic,” which displays a decreasing viscosity with an increasing shear rate. Pseudoplastic non-Newtonian fluids include paints, emulsions, and dispersions of many types. A common household example of a strongly shear thinning fluid is styling gel. Styling gels are aqueous/organic fluids that are primarily composed of water and a vinyl acetate/vinyl pyrrolidone co-polymer (PVP/PA). For example, from a sample of hair gel held in one hand and a sample of corn syrup or glycerin in the other, a person skilled in the art would know that that the hair gel is much harder to pour off the fingers (e.g., a low shear application). However, the gel produces much less resistance to flow when rubbed between the fingers (e.g., a high shear application). Pseudoplasticity can be demonstrated by the manner in which shaking a bottle of ketchup causes the contents to undergo an unpredictable change in viscosity. The force causes it to go from being thick like honey to flowing like water. Other examples of pseudoplastic fluids whose viscosities decrease with increased shear include molten lava, ketchup, whipped cream, blood, and nail polish. It is also a common property of polymer solutions and the aqueous/organic solutions of the present invention in which block co-polymers are dissolved.

When the shear rate is varied, the shear stress does not vary in the same proportion, or even necessarily in the same direction. The viscosity of such fluids thus changes as the shear rate is varied, as shown in FIG. 7. The experimental parameters of the viscosity model all have an effect on the measured viscosity of a non-Newtonian fluid, which is called the “apparent viscosity” of the fluid. Apparent viscosity is accurate only when the explicit experimental parameters are furnished and adhered to. The term “Newtonian fluid” refers to the type of flow behaviour Newton assumed for all fluids. The relationship between shear stress (F′) and shear rate (S) is a straight line. This can be relevant to how a decontamination solution is applied to an area contaminated with one or more toxants.

However, a Newtonian fluid's viscosity remains constant as the shear rate is varied. At a given temperature the viscosity of a Newtonian fluid will remain constant regardless of which speed is used to measure it, as shown in FIG. 7. Newtonian fluids are not as common as the much more complex type of fluids, the “non-Newtonian” fluids.

The term “non-Newtonian” refers to a fluid whose flow properties are not described by a single constant value of viscosity. In a Newtonian fluid, the relation between the shear stress and the strain rate is linear, the constant of proportionality being the coefficient of viscosity. In contrast, for a non-Newtonian fluid, the relationship between the shear stress and the strain rate is nonlinear and can be time-dependent. Therefore, a constant coefficient of viscosity cannot be defined. A ratio between shear stress and rate of strain (or shear-dependent viscosity) can be defined, this concept being more useful for fluids without time-dependent behavior.

There are several types of non-Newtonian flow behavior, which are characterized by the way a fluid's viscosity changes in response to variations in shear rate. The most common types of non-Newtonian fluid are those of greatest interest as potential decontaminant solutions of the present invention. Fluids of this type that can be used for coating and adhering to, or being retained on, surfaces are known as pseudoplastic non-Newtonian fluids. These fluids are also easily aerosolized.

A subcategory of non-Newtonian fluids is known as “thixotropic” fluids. A thixotropic, non-Newtonian fluid undergoes a decrease in viscosity with time, when subjected to constant shear. Modern alkyd and latex paint varieties are often thixotropic and will not run off the painter's brush, but will still spread easily and evenly, since the gel-like paint “liquefies” when brushed out. Other examples of thixotropic fluids whose viscosity decreases with time include, but are not limited to, paint, yogurt, milk, carboxymethyl cellulose, glues, starch, and block co-polymers. The distinction between the behaviors of a shear thinning fluid and a thixotropic fluid is that the former displays decreasing viscosity with increasing shear rate, while the latter displays a decrease in viscosity over time at a constant shear rate.

Such non-Newtonian behavior is further facilitated by synergies between the amphipathic solvents of the aqueous/organic solutions with the amphipathic block co-polymers, which promotes microparticle formation in aerosols and thin film formation on surfaces, thereby facilitating decontamination.

Although the concept of viscosity is commonly used to characterize a material, this characterization alone is inadequate to describe the specific mechanical behavior exhibited by a particular non-Newtonian fluid, which is best studied through several other rheological properties. Such properties can be important in defining the relationship between the stress and strain rate tensors under many different flow conditions. These flow conditions include, but are not limited to, oscillatory shear and extensional flow, which are measured using different devices or rheometers. The properties are better studied using tensor-valued constitutive equations, which are common in the field of continuum mechanics.

In contrast to the Newtonian fluid dynamics of the simple monomeric diols such as the preferred isomers of butanediol, polyols such as block co-polymers can be used in organic aqueous solutions to form non-Newtonian fluids. These fluids are mildly viscous when static, but have low viscosity under shear. Non-Newtonian flow can be envisioned by thinking of any fluid as a mixture of molecules with different shapes and sizes. As they pass by each other, as happens during flow, the size, shape, and interactions of the molecules (e.g., hydrogen bonding) will determine how much force is required to move them. At each specific rate of shear, the alignment of the molecules may be different and more or less force may be required to maintain motion.

In one aspect of the present invention, the polyols used to form non-Newtonian fluids are polymers comprised of many monomeric diols. Examples of preferred polymeric diols are the polyethylene glycols (PEGs), the polypropylene glycols (PPGs), random co-polymers and polyalkylene glycols (PAGs), and block co-polymers. As a general rule, the polymer names are designated by one or more capital letters that represent the oxide used to make the polymer. For example, E represents ethylene oxide (EO), and P is propylene oxide (PO). When the two monomers are used in combination, then the name indicates that both oxides are used, as is the case in the block co-polymers of polyethylene oxide, polypropylene oxide (PEO-PPO). A number following the letters of the name indicate the approximate molecular weight.

It is another aspect of the invention that the viscosities of certain monomeric diols are not satisfactory for use in creating aerosol decoantaminants. An example is monomeric ethylene glycol, which is a Newtonian fluid most temperatures and is too viscous for aerosolization. In contrast, the polyethylene block co-polymers of the present invention have low viscosities even at low temperatures. Monomeric ethylene glycol is representative of all of the ethylene glycols. However, the viscosity is highly non linear with respect to its mole fraction when mixed with water to form one of the aqueous/organic solutions of the present invention, becoming highly viscous at low water fractions. Moreover, such aqueous/organic solutions freeze at temperatures below 40° F., and the water fraction is below 40% by volume, rendering them useless as chemical agent decontamination solutions under extreme weather conditions. Owing to their high valued viscosities, many simple monomeric diols cannot be used at higher concentrations to create solutions that can be aerosolized or sprayed for use in decontamination. Instead, they can only be used in localized surface applications.

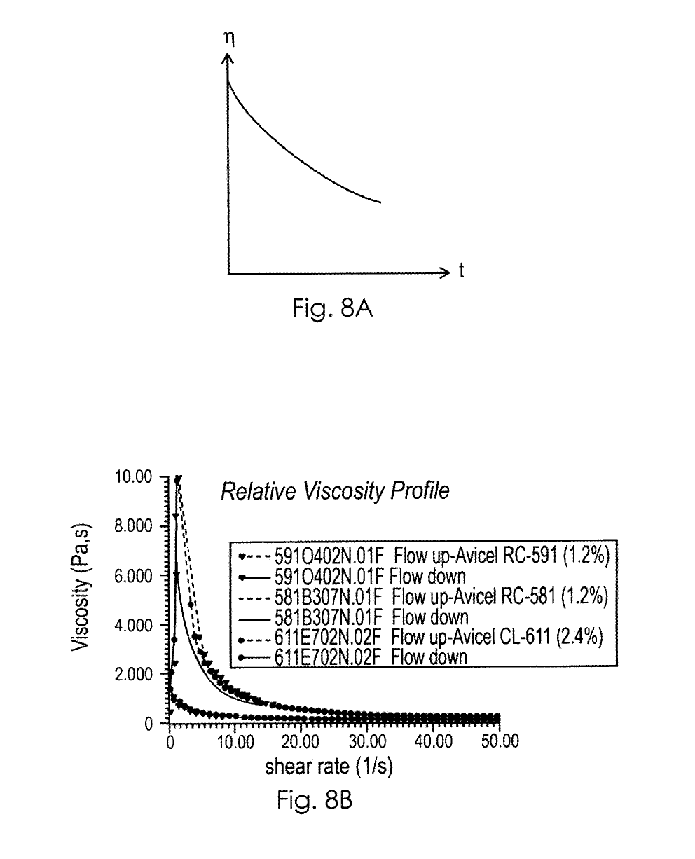

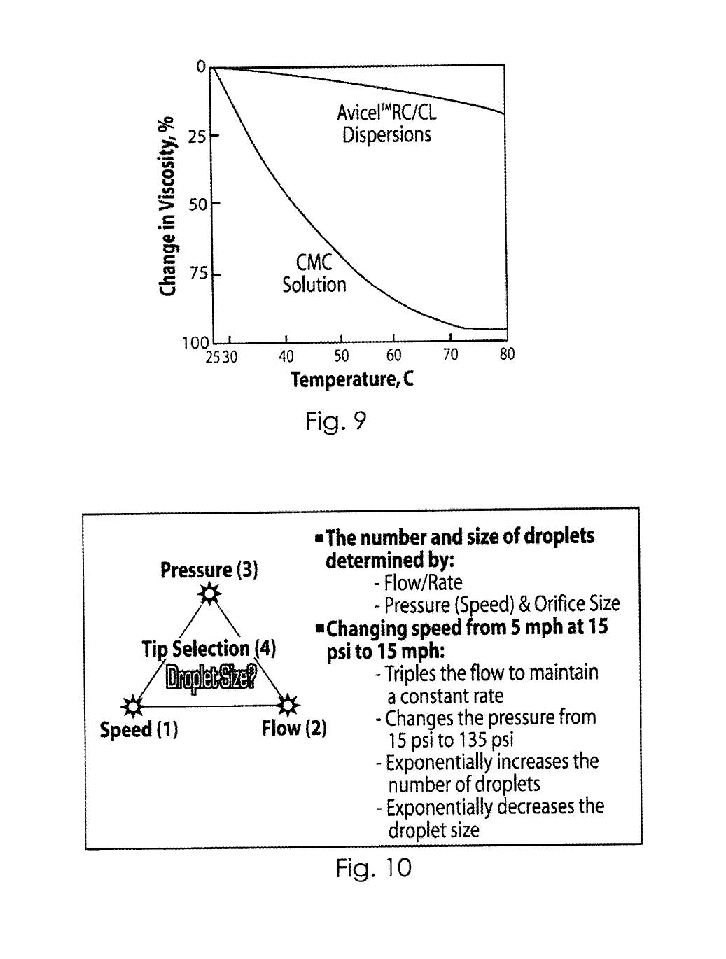

An example of a polymer which can be used to create a thixotropic non-Newtonian fluid is given in FIGS. 8A-8B and 9. Carboxymethylcellulose (CMC) is actually a family of cellulose derivatives with carboxymethyl groups (—CH₂—COOH) bound to some of the hydroxyl groups of the glucopyranose monomers that make up the cellulose backbone. Most CMCs dissolve rapidly in cold water and are mainly used for controlling viscosity without gelling (CMC, at typical concentrations, does not gel even in the presence of calcium ions). As its viscosity drops during heating, it may be used to improve the volume yield during baking by encouraging gas bubble formation. Its control of viscosity allows use as thickener, phase and emulsion stabilizer (for example, with milk casein), and suspending agent. The average chain length and degree of substitution are of great importance; the more-hydrophobic lower substituted CMCs are thixotropic but more-extended higher substituted CMCs can be pseudoplastic. FIG. 8A shows the pseudoplastic behavior of the thixotropic fluid, carboxymethyl cellulose (CMC). FIG. 8B shows a relative viscosity profile of various carboxymethylcelluloses. FIG. 9 relates to the change in viscosity of cellulose solutions due to temperature changes.

The Avicel® RC/CL (microcrystalline cellulose) dispersible celluloses are used in pharmaceutical suspensions, emulsions, nasal sprays, and creams. The wide range of thixotropies, viscosities, gel strengths, and dispersion characteristics of this product line provide unparalleled suspension stability and functional versatility. These polymers are non-Newtonian in solution, however, we have found that their use in decontaminants is largely restricted to topical applications, rather than in sprays, aerosols and fogs.

Another one of the novel discoveries of the present invention is that many polymers form non-Newtonian fluids that are useful in modifying the fluid dynamics of liquids and aerosols. In the preferred formulations, the block co-polymers of the present invention form liquid-in-liquid microemulsions that are used in creating decontaminants that can be used as aerosols or fogs.

Another embodiment of the present invention relates to methods of aerosolizing decontamination solutions by dispersing the solutions through a nozzle. Shear can be induced by air pressure moving fluids through tubing and nozzles. The air pressure used in moving the decontaminant solutions of the present invention through tubing, the tubing itself, and the geometry and operation of the aerosolizing or spray nozzle, all generate shear. Droplet formation, or atomization, begins when a liquid is forced through a hydraulic nozzle under pressure so that the liquid forms a thin sheet that subsequently breaks up into droplets. Each nozzle produces a range of droplet sizes, known as the droplet spectra or drop-size distribution. Droplet size is measured in microns (μm). In general, the range of droplets produced by a nozzle depends on nozzle design. The smallest droplets, ideal for applications such as pesticide control and chemical agent neutralization and suppression can be produced by air atomizing nozzles. The largest droplets, which are ideal for washing and cleaning, can be produced by flat fans.

In order to integrate a nozzle design with a non-Newtonian decontamination solution formulation, certain factors must be considered. Droplet size is both a consequence of the formulation and a determinant of surface coverage at the target surface. Small droplets are more subject to off target drift than larger droplets. Increasing the velocity of the droplet will make it less susceptible to drift. Small drops are more likely to be retained by surfaces than large drops. It has been found in agricultural spraying that very few, if any droplets larger than 200 microns are retained by plant leaves which are difficult to wet. The drops will bounce off the leaf surface, and be deposited on the ground. FIG. 10 shows how the number and size of droplets are determined during aerosolization.

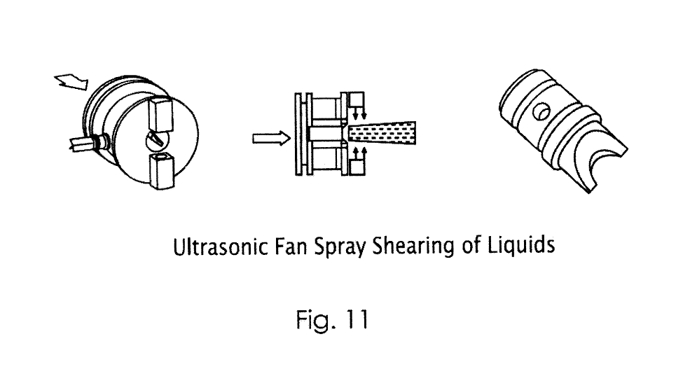

Several of the nozzles used in evaluating the aerosol and fogging properties of the present invention are shown in FIG. 11. The smaller the diameter of the tube through which the solution is delivered and the greater the speed of air flow, the more shear the solution encounters. Any restrictions, reductions in diameter, and/or turns increase the shear. Changing the diameter of a pipe will create both back pressure and internal friction, if the objective is to push the same volume of fluid through a smaller diameter. T-shaped joints, valves and other common assemblies also can increase shear. Further, the more distance a solution has to travel, the more shear it encounters. Finally, the geometry of the nozzle and the turbulence at the nozzle head all can induce shear. All of these factors can change the sizes of the microparticles at the nozzle head.

The preferred form in which the formulations of the present invention are aerosolized is a liquid-in-liquid microemulsion, which is a non-Newtonian fluid, as described above. Conversion of the organic/aqueous solutions of the present invention to block co-polymer microemulsions facilitates dispersal of the decontamination solutions using a range of high performance nozzles including, but not limited to, ultrasonic nozzles. Thus, an additional aspect of the present invention are, collectively, methods of converting organic/aqueous mixtures of the invention to pseudoplastic non-Newtonian solutions for use with a range of high performance nozzles including, but not limited to, ultrasonic nozzles.

Surface active components of the organic/aqueous mixtures are also useful for forming microemulsion fogs that enhance aerosol decontamination by increasing (i) the rates of toxant hydrolysis, (ii) the aerosol persistence of the decontaminating solution when dispersed through high performance nozzles; and (iii) the area covered per unit volume of decontaminant. The surfactant components can also form microemulsion sprays that enhance surface decontamination when dispersed through high performance nozzles. In this case, the surface active components form a thin film that holds the decontamination solution on the surface to be decontaminated. Thus, other embodiments of the present invention include methods of forming microemulsion fogs or sprays when dispersed through high performance nozzles.

One embodiment of the present invention are decontamination solutions with minimum viscosity at the dispersion nozzle, but optimum viscosity for diffusion and retention on the target surface Such formulations produce medium sized droplets with no change in droplet size with speed, pressure, flow fluctuations, and temperature. To compare droplet sizes produced by different nozzle designs, droplet diameters derived using the same assessment method must be used. FIG. 10 shows how droplet size is determined. Viscosity and surface tension are the two main factors that influence droplet size. Generally, as viscosity or surface tension is increased, the forces required to generate droplets increases. This increase in required force results in less energy available for atomization. Hence, viscous liquids or those with high surface tension tend to form more coarse droplets. Moreover, as flow rate increases, an increase in droplet size is also observed. In the case of air atomizers, increasing the shear velocity of the liquid will decrease droplet size. The shear velocity is a function of viscosity.

According to certain aspects of the invention, methods of decontaminating toxants can use a decontamination solution comprising an aqueous/organic solution providing certain capabilities. Specifically, the solution can rapidly dissolve full threat loads of toxants in a homogeneous, isotropic solution; can rapidly dissolve reactive oxygen species or their dry sources in sufficient concentrations to rapidly and completely hydrolyze or otherwise neutralize full threat; can rapidly dissolve loads of toxants in small volume ratios of decontaminant solution to toxant; and can remain a low viscosity liquid with a low vapor pressure from about 125° F. to about −25° F. Further, the solution can also improve the reaction kinetics of decontamination; can minimize the volume ratio of decontamination solution/toxant for greater efficacy; can utilize surfactant/block co-polymer synergies to create non-Newtonian solutions; and can minimize damage to sensitive equipment (e.g., electronics and specialized materials). The solution should also have a sufficiently low vapor pressure and high flash point, to ensure easy and safe shipping, storage, and use. In addition, ease of cleanup and disposal of the solution and the toxant by-products is desirable, as are environmental safety and biodegradability.

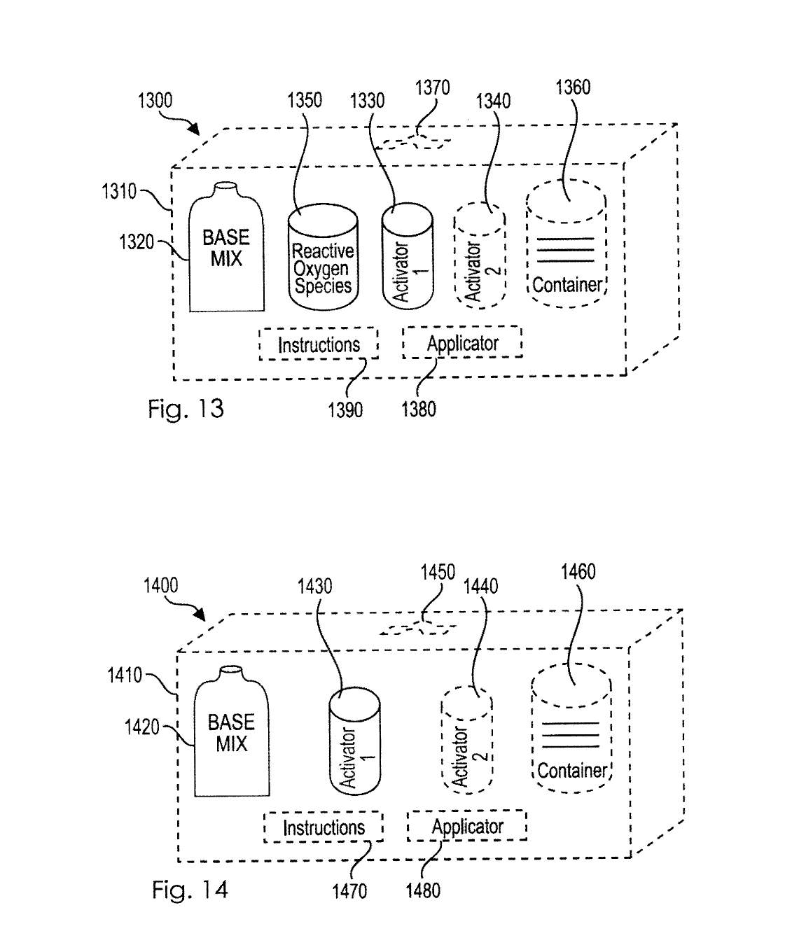

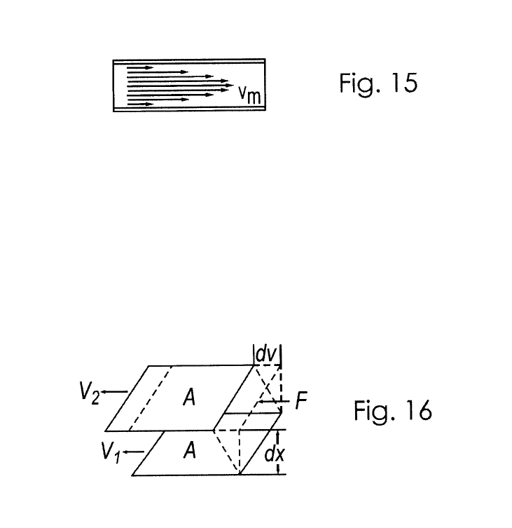

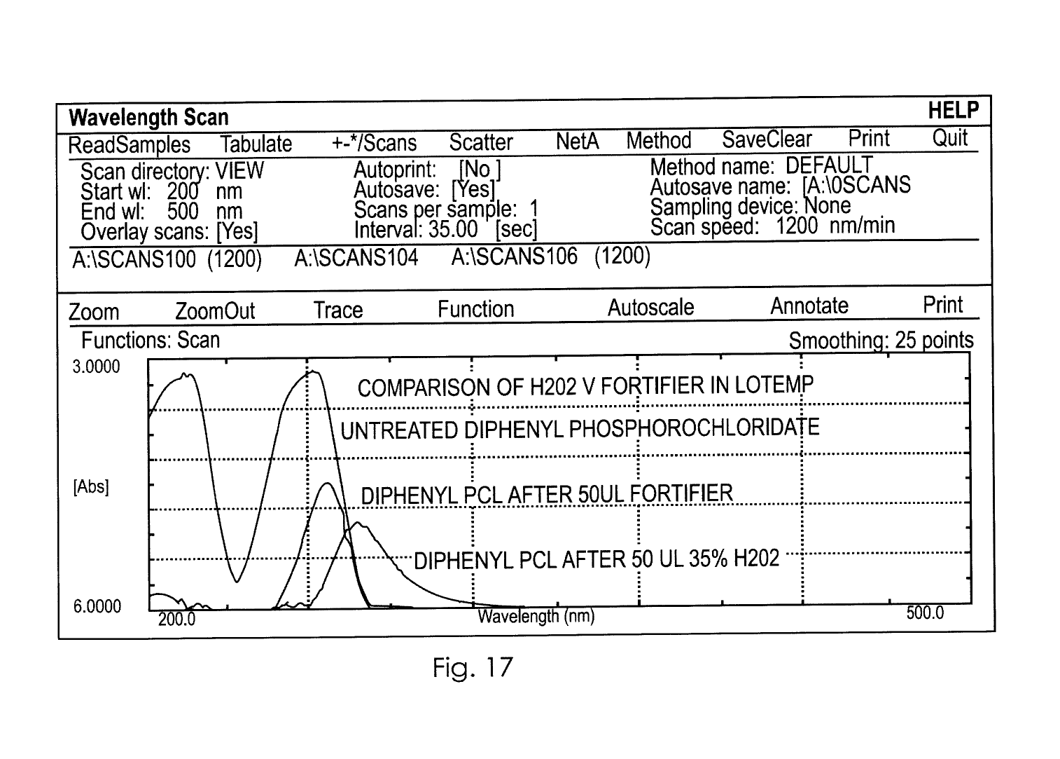

Another aspect of the invention relates to systems comprising the decontaminants of the instant invention. One system configuration for any of the polyol based formulations can comprise four parts: the base mix, at least one liquid activator (which may be two liquid activators), and a dry activator. This format is known as a quaternary system (FIG. 13). Alternative system configurations can comprise three parts (a ternary kit) or two parts (a binary kit). The latter system would include a base solution which contains all of the components except either (i) the reactive oxygen species or its source, or (ii) the liquid activator or dry activator or source thereof. The binary system configuration of the present invention (FIG. 14) has the peroxy acid source(s) dissolved in their stable unactivated states in the base mix, and the activator source, in either its stable liquid or dry source, would be contained in a separate container.

The system components can be routinely supplied in two, three, or four containers, depending upon the system configuration. The components can be mixed on an as-needed basis in the required volumes. In addition, the base mix can be shipped fully diluted with water, or, in the case of the high flash point polyol based formulations, shipped without the water fraction. Certain embodiments of the invention comprise mixed components as a solution ready for deployment. Other embodiments include hardware for dispersal of decontamination solutions as either an aerosol fog or as a spray for surface applications. Other embodiments of the invention use the solutions as fogs or sprays to hydrolyze chemical toxants.

The following examples illustrate concepts related to the present invention.

### Example 1

The melting point of a solution of 9% isopropyl alcohol PA, 9% H₂O₂, and 82% H₂O by volume can be determined. CH₃—CH(CH₃)—OH does not dissociate appreciably and forms an ideal solution. 2 moles of hydrogen peroxide are disproportionate

H₂O₂→H₂O+O₂

so the solute is CH₃—CH(CH₃)—OH. The concentration of CH₃—CH(CH₃)—OH is 8% v/v=80 ml/L; the density of CH₃—CH(CH₃)—OH=0.7855 g/ml; the solution contains 0.7855 g/ml×80 ml=62.84 g; the molecular weight of CH₃—CH(CH₃)—OH=60.10 g/M. 62.84 g/60.10 g/M=1.046 Moles is added to 920 ml water with a density of 1 g/ml. Then ΔT=kfm=1.86° C. kg mol⁻¹×1.137 molal=2.11° C. and the freezing Point=(0−2.11)° C.=−2.11° C.

### Example 2

The amount of glycol (1,2-ethane-diol), C₂H₆O₂ which must be added to 1.00 L of H₂O such that the solution does not freeze above −20° C. may be calculated as follows:

$$m = \frac{Δ T}{{ik}_{f}} = \frac{20.0 ° C .}{1.86 ° C . kg {mol}^{- 1}} = 10.8 mol al$$

where kf (H₂O)=1.86° C. kg mol-1; ΔT=i kf m where i=1. Since 1.0 L has a mass of 1.0 kg, 10.8 mol of ethylene glycol is needed, so 10.8 mol×62 g/mol=670 grains of ethylene glycol. The density of ethylene glycol is 1.1088. Therefore, 670 g/1.1088 g/mL=604 ml, which is dissolved in 1 L=6.04 ml in 10 ml. At this concentration, the viscosity of such a solution renders it unusable for aerosol spraying.

### Example 3

The amount of NaCl that must be added to 1.00 L of H₂O to decrease the freezing point to −20° C. can be calculated:

$$m = \frac{Δ T}{{ik}_{f}} = \frac{20.0 ° C .}{1.86 ° C . kg {mol}^{- 1}} = 5.376 mol al$$

where NaCl(s)→Na+(aq)+Cl−(aq) i=2 and i kf=2 (1.86° C. kg mol⁻¹). Therefore, 5.376 mol×58.44 g/mot=314.17 g NaCl must be added to 1 kg (1 L) of H₂O.

### Example 4

The amount of NaO₂ must be added to 1.00 L of H₂O to decrease the freezing point to −20° C. can also be calculated. In this example, the salts CsO₂, RbO₂, KO₂, and Na₂O₂ were prepared by the direct reaction of O₂ with the respective alkali metal. The O—O bond distance in NaO₂ is 1.33 Å, vs. 1.21 Å in O₂ and 1.49 Å in O₂²⁻. The overall trend corresponds to a reduction in the bond order from 2 (O₂), to 1.5 (O₂⁻), to 1 (O₂²⁻). The alkali salts of sodium are orange-yellow in color and quite stable, provided they are kept dry. Upon dissolution of these salts in water, however, the dissolved decomposes extremely rapidly:

NaO₂(s)→Na+(aq)+O₂⁻(aq) i=2

₂O₂⁻+2H₂O→O₂+H₂O₂+2OH⁻

2H₂O₂→2H₂O+O₂

In this process O₂⁻ acts as a strong Brønsted base, initially forming HO₂. The pKₐ of its conjugate acid, hydrogen superoxide (HO₂, also known as “hydroperoxyl” or “perhydroxy radical”) is 4.88, so that at neutral pH 7 the vast majority of superoxide is in the anionic form.

$${NaO}_{2} ( s ) \to Na + ( aq ) + {O}_{2}^{-} ( aq )$$

$$m = \frac{Δ T}{{ik}_{f}} = \frac{20.0 ° C .}{2 ( 1.86 ° C . kg {mol}^{- 1} )} = 5.376 mol al$$

Therefore, 5.376 mol×54.98 g/mol=295.57 g NaO₂ must be added to 1 kg (1 L) of H₂O.

### Example 5

This Example relates to the ultraviolet absorbance spectra of diphenyl phosphorochloridate (DPCP) and establishing a standard curve for detecting and quantitating hydrolysis of an organophosphate ester, diphenyl chlorophosphate, which is a stimulant for G-class Nerve Agents. For the testing of the G surrogates, certain physical properties were investigated and employed. DPCP has two six-carbon aromatic phenyl rings attached to a single phosphorous atom through an ether bond.

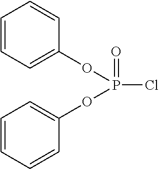

Other properties include the fact that the phenol and phenyl aromatic rings of this chemical are structurally related to benzene; the absorbance spectra of benzene like systems are characterized by E-bands and B-bands; the absorbance spectrum of benzene shows broad absorption bands in the near ultraviolet region between 230 nm and 270 nm; the fine structure arises from vibrational sublevels accompanying the electronic transitions; and the substitution of auxochromic groups on to the benzene ring produces marked changes in the benzene spectrum.

The conversion of phenol to phenolate creates an additional unshared pair of non-bonding electrons available for interaction with the n-electrons of the aromatic nucleus. The availability of these additional non-binding electrons result in a bathochromic shift of the first and second bands. One objective of this example is to establish a standard curve for the detection of diphenyl chlorophosphate. The parameters to detect units vs. molar concentration are as follows: (i) the MW=268.33 g/M; (ii) the LD₅₀ is not available; (iii) the dynamic Range objective=6 logs; (iv) the lower limit of detection objective=4.8×10⁻⁶ M/L=4.8 μMolar=6.192 ng/ml; (v) the molar extinction coefficient 260 nm≈459 L mole⁻¹ cm⁻¹ in one solution of the invention; and (vi) the molar extinction coefficient 220 nm≈6,000 L mole⁻¹ cm⁻¹.

The detection technologies evaluated in this example include absorbance spectroscopy using cuvette sampling; reversed phase HPLC with an absorbance detector; HPLC/Mass Spectroscopy; gas chromatography with a flame ionization detector; and solid phase sampling with HPLC.

### Example 6

This Example relates to the generation of a standard curve for the detection of the organophosphate ester DPPC, which structure and characteristics are given above. The following equations are useful for the determination of hydrolysis of a biological or chemical warfare agents.

A = ε l c where the units of c are L mole⁻¹ cm⁻¹.
assume c = 0.1% solution = 4.8 × 10⁻³ Molar
assume that the pathlength = 1 cm and that A₂₆₀ at c = 2.2 OD
$then ɛ = \frac{2.2 OD}{1 \times 0.458 \times {10}^{- 3}} = 0.4583 \times {10}^{3} = 459 L {mole}^{- 1} {cm}^{- 1}$

| then if l = | OD₂₂₀ | | | OD₂₆₀ | | | Concentration DPCP | % solution |
|---|---|---|---|---|---|---|---|---|
| 1 cm | 28,758 | | | 2200.000 | | | 4.8 × 10⁰ M/L | neat solution |
| 1 cm | 2,875.8 | | | 220.000 | | | 4.8 × 10⁻¹ M/L | 10.0% |
| 1 cm | 287.58 | {close oversize brace} | [symbol] | 22.000 | | | 4.8 × 10⁻² M/L | 1.0% |
| 1 cm | 28.758 | | | 2.200 | | | 4.8 × 10⁻³ M/L | 0.1% |
| 1 cm | 2.876 | | | 0.220 | | | 4.8 × 10⁻⁴ M/L | 0.01% |
| 1 cm | 0.287 | | | 0.022 | {close oversize brace} | * | 4.8 × 10⁻⁵ M/L | 0.001% |
| 1 cm | 0.028 | {close oversize brace} | * | 0.0022 | | | 4.8 × 10⁻⁶ M/L | 0.0001% |
| 1 cm | 0.0028 | | | — | | | 4.8 × 10⁻⁷ M/L | 0.00001% |

[symbol]

= Dynamic range in a 0.001 cm pathlength at this wavelength
* = Dynamic Range in a 1 cm pathlength at this wavelength

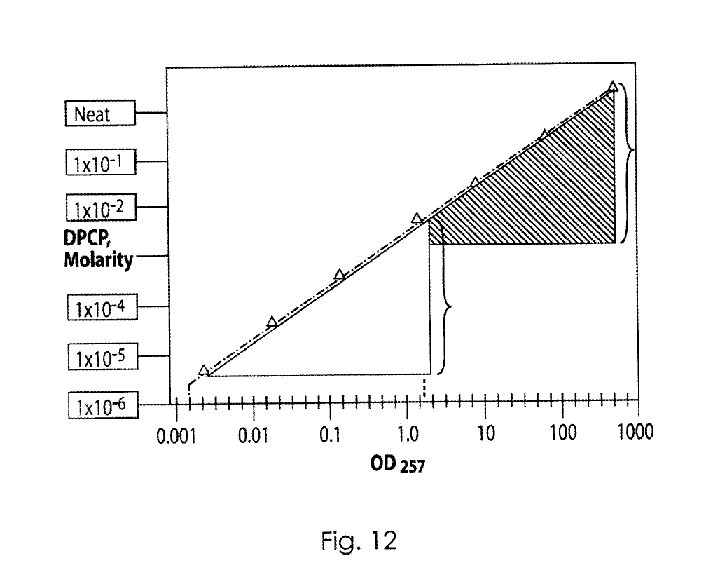

The standard curve generated using the data above is seen in FIG. 12 and created using Beers Law.

### Example 7

This Example relates to the hydrolysis of an organophosphate ester by reactive oxygen species in an organic/aqueous solution of the present invention. The diagram shown in FIG. 22 shows the results of the hydrolysis of an organophosphate ester by a reactive oxygen species in an organic/aqueous solution as described herein.

### Example 8

The following decontaminant formulation is an another example of the invention in the quaternary kit configuration. The formulation has been used as the reference standard or “baseline” formulation for decontamination efficacy and stability (aka “Shelf Life”) against which all other decontaminant formulations and kit configurations have been compared:

| | % by Volume | Moles/L |
|---|---|---|
| Part I—Base Mix (Solvent) | | |
| 2,3-Butanediol | 52.50 | 5.476 |
| 1-Hexanol | 5.20 | 0.398 |
| Neat Hydrogen Peroxide | 7.90 | 3.279 |
| Water | 33.90 | 1.883 |
| Block Co-Polymer (Pluronic F-127) | 0.50 | 0.00007 |
| | 100.00 | |
| Part II—Dry Activator | | |
| Recrystallized TAED | 100.00 | 0.867 |
| Part III—Liquid Activator 1 | | |
| Peroxyacetic Acid | 39.00 | 0.297 |
| Acetic Acid | 45.00 | |
| Hydrogen Peroxide | 6.00 | |
| | 100.00 | |
| Part IV—Liquid Activator 2 | | |
| Sodium Hydroxide Solution (5.5M Solution) | 100.00 | 0.550 |

### Example 9

The following decontaminant formulation is another example of the invention in the binary kit configuration.

| | % by Volume | Moles/L |
|---|---|---|
| Part I—Base Mix | | |
| 2,3-butanediol | 52.50 | 5.476 |
| n-hexanol | 5.20 | 0.3982 |
| neat H₂O₂ | 7.90 | 3.279 |
| neat peroxyacetic acid | 2.00 | 0.434 |
| TAED | 1.00 | 0.367 |
| Water + acetic Acid + sulfuric acid | 32.90 | 1.663 |
| Block co-polymer | 0.50 | 0.00007 |
| Part II—Activator | | |
| Sodium Hydroxide Solution (5.5M Solution) | 100.00 | 0.550 |

Since the formulation of the binary reference kit differs significantly from the reference standard in the quaternary reference kit, this binary formulation was established as the reference standard for all other decontaminant formulations and kit configurations that are prepared in binary kit configurations.

One important aspect of the reference standard formulations given above is that they are unbuffered formulations. In these formulations, chemical activation is achieved by the initial pH change of the solution to the alkaline by the addition of concentrated base, which in this case is 5.5 M NaOH. Under such conditions, the amount and rate of perhydrolysis are not regulated by buffering capacity of the solution. This enabled evaluation of different buffers and buffer concentrations to identify (i) the optimum rate of chemical activation by perhydrolysis to identify the preferred mode of the invention; and (ii) the optimum formulation for maximum shelf life of the reactive oxygen species and activator.

A second aspect of the reference standard formulations is that they contain a molar excess of hydrogen peroxide relative to the molar amount of TAED. Under such conditions, the amount and rate of perhydrolysis are not regulated by buffering capacity of the solution. This enabled evaluation of different buffers and buffer concentrations to optimize the rate of chemical activation of perhydrolysis by different buffers to identify the preferred mode of the invention. In the present invention, the reference standards were established for the purpose of creating formulations with optimum: (i) rates of perhydrolysis of the percarboxylic acid sources; (ii) stocihiometries of activator and reactive species for maximum activated life (pot life); and (iii) optimum efficacy against known threat loads of toxants. Establishing such reference standards against which the efficacy of other formulations could be compared was an important invention.

In the preferred embodiments of the present invention, the method of chemical activation is not a simple pH adjustment with a base, but instead is an activation by a buffering system in which the buffering capacity is used to establish the quasi-steady state equilibrium of reactive species production as described above. A second aspect of the preferred embodiment of the invention is that the stoichiometry of the activator and reactive oxygen species is optimized to provide the greatest rate and efficacy in reducing threat loads of chemical or biological toxants. In yet a third aspect of the preferred embodiment of the invention, the kit configuration is binary. It is yet a fourth aspect of the preferred embodiment of the invention that such a kit would have a small logistical footprint. A fifth aspect of the preferred embodiment of the invention is that it can be readily aerosolized as a spray. A sixth aspect of the preferred embodiment of the invention is a formulation that is easy and safe to ship, store, use, and cleanup. Finally, a preferred embodiment of the invention is that the formulation is environmentally safe to use and has excellent materials compatibility.

### Example 10

The invention also relates to a quaternary kit 1300 comprising a kit container 1310 for the components to prepare a chemical or biological decontaminant solution. The kit container 1310 can be, but is not limited to, a suitcase, a box, or a bucket-type kit container. The kit container 1310 can be opaque or transparent. If transparent, an end user is able to determine if the kit 1300 contains the exact components desired. The kit can comprise at least at least one polar organic amphipathic solvent Base Mix 1320, at least one dry or liquid activators or sources thereof, 1330 and 1340, and at least one liquid or dry reactive oxygen species, 1350. The solvent can be a polar aprotic solvent, a polar-protic solvent, or combinations thereof. Further, the solvent can be a nitrile, a ketone, an aldehyde, a carboxylic acid, an amide, a furan, an alkanol or a polyol. The volume fraction of water in decontaminant solution can range from about 25% to about 80% or from about 25% to about 75%, and the pH of the solution can be less than or equal to about 8.5. More specifically, the polar amphipathic solvents can be butanediol, an isomer of butanediol, 1-hexanol, a linear or branched-chain alcohol with 1 to 15 carbons, an n-alcohol, a butoxy-alcohol, or a combination thereof. The active oxygen species can be tetraacetylethylenediamine (TAED) or tetraacetylmethylenediamine (TAMD). Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

The at least one liquid or dry activator 1330, 1340 of the quaternary kit can comprise any peroxide or persulfate which is the source in the decontaminant solution of hydroxyl radicals, hydroxyl ions, super oxides, or other oxidizers, which perhydrolyze the reactive oxygen species source. In addition, the at least one reactive oxygen species 1350 can comprise peroxyacetic acid or its source, a first activator 1330 that is a hydrogen peroxide activator and a second activator 1340 that comprises a buffer system, which includes, but is not limited to, carbonate or sodium hydroxide based buffer systems. The kit of the invention can further comprise a block co-polymer, which would be pre-mixed with the base mix solvent 1320, wherein the block co-polymer can be ethylene oxide and propylene oxide co-polymer that terminates in primary hydroxyl groups.

The quaternary kit can also comprise a container 1360 for mixing the solution, the at least one solvent 1320, at least one dry or liquid activator or source thereof 1330, 1340, and at least one reactive oxygen species 1350, which can be combined together into a decontamination mixture, along with means for physically associating the decontamination mixture with the toxant. The quarternary kit 1310 can use a wide variety of materials to store the components of the system, such as vessels made of but not limited to, metal, clear glass, colored glass or plastic.

The means for physically associating the decontamination mixture with the toxant can comprise an applicator 1380 including, but not limited to, an aerosolization nozzle. The kit container 1310 can comprise a handle 1370 for carrying the kit container with components. The kit container 1310 can also comprise wheels for ease of transport (not shown). Further, the kit can comprise instructions for use 1390.

### Example 11

The invention also relates to a binary kit 1400 comprising a kit container 1410 for the components to prepare a chemical or biological decontaminant solution. The kit container 1410 can be, but is not limited to, a suitcase, a box, or a bucket-type kit container. The kit container 1410 can be, but is not limited to, a suitcase, a box, or a bucket-type kit container. The kit container 1410 can be opaque or transparent. If transparent, an end user is able to determine if the kit 1400 contains the exact components desired. The kit, or system, 1400 can comprise: (i) a liquid Base Mix 1420, comprising at least at least one polar organic amphipathic solvent and a reactive oxygen species or its source, and (ii) at least one dry or liquid activator or source thereof 1430. The solvent(s) can be a polar aprotic solvent, a polar-protic solvent, or combinations thereof. Further, the solvent(s) can be a nitrile, a ketone, an aldehyde, a carboxylic acid, an amide, a furans, an alkanol, or a polyol. The volume fraction of water in decontaminant solution can range from about 25% to about 75%, and the pH of the solution can less than or equal to about 8.5. More specifically, the polar amphipathic solvents can be butanediol, an isomer of butanediol, a linear or branched-chain alcohol with 1 to 15 carbons, an n-alcohol, a butoxy-alcohol, other polyols, or a combination thereof. The active oxygen species can be tetraacetylethylenediamine (TAED) or tetraacetylmethylenediamine (TAMD). Alternatively, the at least one reactive oxygen species can comprise peroxyacetic acid or its source. Alternatively, the pH can be maintained at less than or equal to about 8.0. The invention is active against all agents as pH values between about 7.0 to about 10.5; the buffer capacity is more effective at pH values between about 8.0 to about 9.0. However, for maximum active life, the preferred embodiment is maintained a pH values from about 8.0 to about 8.5. The pH can further be maintained at a pH of about 8.5.

The least one liquid or dry activator 1430 of the binary kit can comprise any peroxide or source thereof which is the source in the decontaminant solution of hydroxyl radicals, hydroxyl ions, super oxides, and which perhydrolyze the reactive oxygen species source. In addition, the binary kit can comprise a first activator 1430 (e.g., a hydrogen peroxide activator) and a second activator 1440 (e.g., a carbonate or sodium hydroxide based buffer system). The kit of the invention can further comprise a block co-polymer, which would be pre-mixed with the Base Mix 1420, wherein the block co-polymer can be an ethylene oxide and propylene oxide co-polymer that terminates in primary hydroxyl groups.

The binary kit 1410 can use a wide variety of materials to store the components of the system, such as vessels made of, but not limited to, metal, clear glass, colored glass, or plastic.

The binary kit can also comprise a mixing container 1440 or an automated system for simultaneous mixing and activation of the Base Mix 1420 containing at least one reactive oxygen species with the at least one dry or liquid activator or source thereof 1430, which can be combined to create a decontamination mixture, along with means for physically associating the decontamination mixture with the toxant. The means for physically associating the decontamination mixture with the toxant can comprise an applicator 1480 including, but not limited to, an aerosolization nozzle. The kit container 1410 can comprise a handle 1450 for carrying the kit container with components. Further, the kit can comprise instructions for use 1470.

One binary kit of the present invention which meets all of the requirements of the preferred embodiment has the following formulation:

| | % by Volume | Moles/L |
|---|---|---|
| Part I—Base Mix | | |
| 1,3-butanediol | 52.50 | 5.574 |
| 2-butoxy ethanol | 5.20 | 0.3808 |
| TAED | 1.00 | 0.434 |
| Water | 32.90 | 12.5 |
| Block co-polymer | 0.50 | 0.00007 |
| Part II—Dry Activator | | |
| Sodium percarbonate (dry) | 100.00 | 1.445 |

The foregoing descriptions of the invention are intended to be illustrative and not limiting. Those skilled in the art will appreciate that the invention can be practiced with various combinations of the functionalities and capabilities described above, and can include fewer or additional components than described above. Certain additional aspects and features of the invention are further set forth below, and can be obtained using the functionalities and components described in more detail above, as will be appreciated by those skilled in the art after being taught by the present disclosure.

Although the present invention has been described with reference to specific exemplary embodiments, one of ordinary skill in the art would know that various modifications and changes may be made to these embodiments without departing from the broader spirit and scope of the invention. Accordingly, the specification and drawings are illustrative, rather than restrictive.

1. A system for decontaminating chemical and biological agents comprising:
a water-soluble polar organic amphipathic solvent;
an activator that provides a buffering system to establish and maintain a pH of about 8.0 to about 8.5; and
a reactive oxygen species (ROS);
wherein, upon mixing the solvent, the activator, and the ROS with water, a single-phase aqueous decontamination solution is formed, the solution produces and maintains a sufficient amount of singlet oxygen molecules and/or percarboxylate anions to decontaminate a threat load of toxant.

2. The system of claim 1, wherein the polar organic amphipathic solvent is selected from the group consisting of butanediol, isomers of butanediol, 1-hexanol, a linear or branched-chain alcohol with from 1 to 15 carbons, butoxy-alcohol, and combinations thereof.

3. The system of claim 2, wherein the activator is a peroxide obtained from a peroxide source selected from the group consisting of sodium percarbonate, sodium perborate, urea peroxide, and sodium peroxide, and combinations thereof, wherein the activator is a source of a perhydrolyzing agent selected from the group consisting of a hydroxyl radical, a hydroxyl ion, a hydroperoxide anion, and a superoxide.

4. The system of claim 3, wherein the reactive oxygen species is selected from the group consisting of tetraacetylethylenediamine (TAED) and tetraacetylmethylenediamine (TAMD).

5. The system of claim 1, further comprising a block co-polymer, wherein the block co-polymer is a polyethylene oxide and polypropylene oxide co-polymer that terminates in primary hydroxyl groups.

6. The system of claim 5, further comprising an aerosolization nozzle for physically associating the decontamination solution with the toxant.

7. The system of claim 1, further comprising a container for mixing the polar organic amphipathic solvent, the activator, the reactive oxygen species, and water.

8. A method of decontaminating a chemical or biological toxant, the method comprising:
mixing a water-soluble polar organic amphipathic solvent, an activator that provides a buffering system to establish and maintain a pH of about 8.0 to about 8.5, and a reactive oxygen species with water to form a single-phase aqueous decontamination solution; and
physically associating the decontamination solution with the toxant;
wherein the solution produces and maintains a sufficient amount of singlet oxygen molecules or percarboxylate anions, thereby decontaminating a threat load of toxant.

9. The method of claim 8, further comprising:
testing for the presence of the toxant; and
repeating the steps of mixing the water-soluble polar organic amphipathic solvent, the activator, and the reactive oxygen species with water to form a single-phase aqueous decontamination solution; and
physically associating the decontamination solution with the toxant until the level of the toxant is reduced by at least 99.4%,
wherein the solution produces and maintains a sufficient amount of singlet oxygen molecules or percarboxylate anions, thereby decontaminating a threat load of toxant.

10. The method of claim 9, wherein the polar organic amphipathic solvents are selected from the group consisting of butanediol, isomers of butanediol, 1-hexanol, a linear or branched-chain alcohol with 1 to 15 carbons, butoxy-alcohol, and combinations thereof.

11. The method of claim 10, wherein the activator is a peroxide obtained from a peroxide source selected from the group consisting of sodium percarbonate, sodium perborate, urea peroxide, and sodium peroxide, and combinations thereof, wherein the activator is a source of a perhydrolyzing agent selected from the group consisting of a hydroxyl radical, a hydroxyl ion, a hydroperoxide anion, and a superoxide.

12. The method of claim 11, wherein the reactive oxygen species is selected from the group consisting of tetraacetylethylenediamine (TAED) and tetraacetylmethylenediamine (TAMD).

13. The method of claim 8, wherein the decontamination solution further comprises a block co-polymer, wherein the block co-polymer is ethylene oxide and propylene oxide co-polymer that terminates in primary hydroxyl groups.

14. The method according to claim 13, further comprises physically associating the decontamination solution with the toxant by dispersing the decontamination solution with an aerosolization nozzle.

15. The method of claim 8, wherein the decontamination is conducted at a temperature of between about −35° C. and about 140° C.

16. The method of claim 14, wherein the decontamination is conducted at a temperature of between about −25° C. and about 125° C.
